(19) United States
(12) Patent Application Publication
(10) Pub. No.: US 2016/0195509 A1
(43) Pub. Date: Jul. 7, 2016

(54) MEMS IMPLEMENTATION FOR DETECTION OF WEAR METALS

(71) Applicant: MASTINC., New York, NY (US)

(72) Inventors: Brian JAMIESON, Severna Park, MD (US); Michael DICKERSON, Frederick, MD (US); Steven VAN FLEET, Lagrangeville, NY (US)

(21) Appl. No.: 14/989,587

(22) Filed: Jan. 6, 2016

Related U.S. Application Data
(60) Provisional application No. 62/100,201, filed on Jan. 6, 2015.

(51) Int. Cl. G01N 33/28; G01N 21/71; G01N 21/67

(52) U.S. Cl. CPC G01N 33/2858; G01N 33/2888; G01N 21/67; G01N 21/718; G01N 2201/0221

(57) ABSTRACT

This invention relates to analyzing elements, including metals in mechanical systems. The invention therefore allows for detecting wear elements, such as metals, for example, in lubricants to determine whether the mechanical system is deteriorating, or even approaching failure. The invention relates to an integrated micro-electromechanical (MEMS) apparatus, and methods for using this apparatus.

The present application claims the benefit of U.S. provisional patent application No. 62/100,201, filed Jan. 6, 2015, of which is incorporated herein by reference.

### FIELD OF THE INVENTION

This invention relates to analyzing elements, including metals in mechanical systems. The invention therefore allows for detecting wear elements, such as metals, for example, in lubricants to determine whether the mechanical system is deteriorating, or even approaching failure. The invention relates to an integrated micro-electromechanical (MEMS) apparatus, for example, laser-induced breakdown spectroscopy (LIBS) apparatus, Selective Arrayed Waveguide Spectrometer, or spark-induced breakdown spectroscopy, and methods for using this apparatus.

### BACKGROUND OF THE INVENTION

The conditions of lubricating fluids are often detected using a static, periodic approach, typically requiring removing fluid from the system, often by extracting a sample of the fluid to send to testing laboratories around the world, which have established procedures and methods to measure a number of aspects of the lubricating fluid, including historical time-series of various parameters. It is common practice to apply such time-based longitudinal monitoring of the fluid to detect changes over time to gain an understanding of the changes in performance within the closed environment. For example, the presence of specific particles at increasing concentrations can indicate levels of wear and performance of certain underlying components within the system being lubricated.

This testing typically measures changes in characteristics of the fluid over time, including detecting changes and deterioration of underlying lubricating fluid and additives and the detection of normal (expected) and abnormal (unexpected) “wear” of the moving parts due to normal operation. Static samples are usually sent to a facility that performs a number of tests, including detecting the presence of foreign materials and objects. In some cases, such as when the lubrication fluid is changed, the lubrication filter is commonly sent as well as the oil for testing and detailed analysis. For both the sample and the filter, this is a destructive “tear down” analysis—such that the filter and the sample are not returned to service, but evaluated and subsequently removed. Tests typically performed in the laboratory include detection of metallic and non-metallic particles, presence of water or other non-lubricant liquids, carbon soot and other components, and in some cases, verification that the underlying chemistry of the lubricant is still intact. A written (or electronic) report is generated and transmitted to the stakeholder upon completion of the testing. Results typically take days or weeks from extraction to stakeholder review.

A number of low-cost lubricating fluid measurement products and techniques are available—including a consumer static “check” of a motor oil sample which measures the changes in electrical impedance characteristics (electrical capacitance and resistance when a small electrical source is applied across the sensor where a sufficient sample size of the lubricant bridges the sensor electrode across to the detector). This approach performs a single-dimensional measurement of oil sump fluid properties at a point in time in the evolution of the oil (i.e. a static measurement), providing insight only when the operator manually extracts a sample of oil to be tested and only indicates changes in the electrical properties should the data be appropriately logged and tracked over time. This approach has many drawbacks including the interval sampling (only when the operator makes a measurement), as well as the potential for counteracting forces from the presence of multiple contaminants introduced into the fluid to mask the true state/condition of the lubricant. As an example, in the case of an automobile engine, the normal operation of the combustion engine will produce carbon by-products as a result of the operation of the engine (this is what discolors the oil). If a vehicle were producing only this carbon “soot” the resistance would change (increase) due to the introduction of the soot. If at the same time, the engine were undergoing adverse “wear” to the extent that small metallic particles were produced as an abnormal condition across the internal moving parts, these particles would decrease the resistance, as metal is a better conductor over the base lubricant. In the case where both soot and metallic particles were being produced at the same time, they could partially or completely cancel out some or all the measurable effects—thus providing a false indication of the true condition of the lubricant and underlying engine.

Lubricants are designed to perform beyond their stated range and are further enhanced through the addition of “additives” to extend the lifetime and safety margin of the fluid. Understanding the lubrication longevity is crucial for the safe operation of the system. Replacement of the fluid is performed typically at very conservative (i.e. short) recommended intervals, providing a wide safety margin for the operator. In general, lubricants can operate for significantly longer intervals, or in the case of specific equipment operating in harsh environments (e.g. military equipment used on the battlefield or in mining operations, etc.) may require a more aggressive replacement cycle. It is important to determine when the lubricating fluid cannot continue to perform according to specifications determined by the equipment/system manufacturers. As long as the lubricating fluid is within the safe margin of operation, it may operate indefinitely and not need to be exchanged or replaced with fresh lubricating fluid.

Providing a more precise measure of the fluid's performance can maximize the lifetime of both the lubricant and the equipment the lubricant is protecting. As the cost of the equipment and the hydrocarbon lubricant increase, so does the value of providing both a longer and more precise lifetime of the lubricant and early detection and notification of pending equipment performance deterioration (including motor, filter, and other components in the system, etc.). This approach can potentially save lives when critical equipment failures are detected in advance. In addition, should the fluid fail and contribute to the equipment breaking down, this system potentially eliminates the resources required and time lost to repair/replace the underlying/broken equipment. This approach also avoids the loss of service and resources required to complete oil changes more often than actually needed.

Automotive oil lubricates moving engine parts, extending engine life and improving fuel efficiency. The decision to change oil is usually made on the basis of accumulated engine hours or calendar days, with little regard to the actual state of the motor oil. Careful and more continuous monitoring of the state of engine oil allows a more strategic approach to oil changes, accelerating the timing of oil changes when it is needed and delaying it when it is not. In addition, the oil can be seen as the “blood” of the engine, carrying important information about wear and abnormal conditions of the parts of the engine with which it makes contact. For example, the presence of copper in motor oil can indicate abnormal wear of valve train bushings, while large quantities of silicon (which is highly abrasive to engine surfaces) could arise from the ingestion of dirt or particles from breathers or other external sources. This knowledge, if available promptly, could allow early intervention to fix engine problems before they arise or escalate causing further catastrophic damage. In addition, modern engine oils typically contain additives that improve the lubricating properties of the oil through custom and proprietary chemistries. Monitoring changes to the elemental profile of these additive packages could improve understanding of their performance and allow for more calibrated oil changes. LIBS is a recognized approach for performing elemental analysis in the laboratory, however typical equipment is far too delicate, large and costly to function as a practical automotive sensor.

### SUMMARY OF THE INVENTION

The present invention fulfils the needs identified above.

In embodiments, the invention encompasses to an integrated micro-electromechanical (MEMS) spectrometer, for example, laser-induced breakdown spectroscopy (LIBS) apparatus or spark-induced breakdown spectroscopy (SIBS) incorporating a selective arrayed waveguide spectrometer, and methods for using this apparatus for detecting a wear element in a liquid is provided.

Suitably, the apparatus comprises a MEMS substrate form factor, a laser integrated with the MEMS substrate form factor, an optical fiber or free space optical elements (e.g., lenses) configured to transmit a laser pulse from the laser to the liquid and to generate a plasma, an optical fiber or free space optical elements (e.g., lenses) configured to transmit light emitted by the plasma, a spectrometer configured to measure a spectrum of light emitted by the plasma and thus produce data regarding the wear element, and electronics for transmitting the data regarding the wear element.

In embodiments, the liquid is an oil-based lubricant, including for example, an automotive lubricant, a marine lubricant, an aircraft lubricant, an industrial device lubricant, a compressor lubricant, and a wind turbine lubricant.

Suitably, the laser is an IR laser, which may be frequency doubled or quadrupled into the visible or UV portion of the electromagnetic spectrum, and in embodiments is a sub-nanosecond pulse laser.

In exemplary embodiments, the form factor is between about 30 cm³ and about 100 cm³.

Suitably the wear element that is detected is selected from, but not limited to, Na, Mg, Al, Si, Mn, Fe, Ni, Cu, Zn, and Mo. In exemplary embodiments, the wear element in the liquid is detected at a level of between 0.1 and 200 parts per million.

Also provided are systems comprising the MEMS LIBS apparatus as described herein and further comprising a receiver unit remotely located from the MEMS form factor.

In additional embodiments, the invention encompasses a machine comprising the MEMS LIBS apparatus as described herein is also provided, including for example, a machine such as a car, a truck, a boat, a ship, an aircraft, an industrial machine, a compressor, and a wind turbine.

Also provided are methods of detecting wear elements in a liquid, comprising providing a liquid sample, contacting the liquid sample with a laser pulse to generate a plasma (e.g., breakdown means), and detecting one or more wear elements in the plasma with laser-induced breakdown spectroscopy, wherein the liquid is contacted with a laser that is integrated into a MEMS form factor.

Also provided are methods of detecting wear elements in a liquid, comprising providing a liquid sample, contacting the liquid sample with a spark to generate a plasma, and detecting one or more wear elements in the plasma with spark-induced breakdown spectroscopy, wherein the liquid is contacted with a spark that is integrated into a MEMS form factor.

In additional embodiments, the invention encompasses an integrated micro-electromechanical (MEMS) laser-induced breakdown spectroscopy (LIBS) apparatus for detecting a wear element in a liquid is provided. The apparatus suitably comprises a MEMS substrate form factor, a laser integrated with the MEMS substrate form factor, one or more focusing optics or reflectors, a microfluidic flow channel comprising the liquid, collection optics to gather light emitted by the plasma generated by the laser and suitably to direct light to the entrance slit of a spectrometer, and a spectrometer to measure the spectrum of the light emitted by the plasma generated by the laser and generate data regarding the wear element.

In additional embodiments, the invention encompasses an integrated micro-electromechanical (MEMS) spark-induced breakdown spectroscopy (SIBS) apparatus for detecting a wear element in a liquid is provided. The apparatus suitably comprises a MEMS substrate form factor, a high voltage source including electrodes integrated with the MEMS substrate form factor, one or more focusing optics or reflectors, a microfluidic flow channel comprising the liquid, collection optics to gather light emitted by the plasma generated by the spark and suitably to direct light to the entrance slit of a spectrometer, and a spectrometer to measure the spectrum of the light emitted by the plasma generated by the spark and generate data regarding the wear element.

In still further embodiments, the invention encompasses an integrated micro-electromechanical (MEMS) laser-induced breakdown spectroscopy (LIBS) apparatus for detecting a wear element in a liquid is provided. Suitably, the apparatus comprises a porous filter, a laser focused on the porous filter, a plunger for drawing a liquid into the porous filter, collection optics to gather light emitted by the plasma generated by the laser and suitably to direct light to the entrance slit of a spectrometer, and a spectrometer configured to measure the spectrum of the light emitted by the plasma generated by the laser and generate data regarding the wear element.

In still further embodiments, the invention encompasses an integrated micro-electromechanical (MEMS) spark-induced breakdown spectroscopy (SIBS) apparatus for detecting a wear element in a liquid is provided. Suitably, the apparatus comprises a porous filter, a high voltage source including electrodes integrated on the porous filter, a plunger for drawing a liquid into the porous filter, collection optics to gather light emitted by the plasma generated by the spark and suitably to direct light to the entrance slit of a spectrometer, and a spectrometer configured to measure the spectrum of the light emitted by the plasma generated by the spark and generate data regarding the wear element.

In additional embodiments, the invention encompasses an integrated micro-electromechanical (MEMS) laser-induced breakdown spectroscopy (LIBS) apparatus for detecting a wear element in a liquid is provided. The apparatus suitably comprises a MEMS form factor, a laser, an apparatus for ejecting a droplet of the liquid into a focal point of the laser, collection optics to gather light emitted by the plasma generated by the laser and suitably to direct light to the entrance slit of a spectrometer, and a spectrometer to measure the spectrum of the light emitted by the plasma generated by the laser and generate data regarding the wear element.

In additional embodiments, the invention encompasses an integrated micro-electromechanical (MEMS) spark-induced breakdown spectroscopy (SIBS) apparatus for detecting a wear element in a liquid is provided. The apparatus suitably comprises a MEMS form factor, a voltage source including electrodes to generate a spark, an apparatus for ejecting a droplet of the liquid into the voltage source, collection optics to gather light emitted by the plasma generated by the spark and suitably to direct light to the entrance slit of a spectrometer, and a spectrometer to measure the spectrum of the light emitted by the plasma generated by the spark and generate data regarding the wear element.

In further embodiments, the invention encompasses an integrated micro-electromechanical (MEMS) laser-induced breakdown spectroscopy (LIBS) apparatus for detecting a wear element in a liquid is provided. In embodiments, the apparatus comprises a MEMS form factor, a laser, an apparatus for focusing a stream of the liquid into a focal point of the laser, and collection optics to direct light to the entrance slit of a spectrometer, and a spectrometer to measure the spectrum of a plasma generated by the laser and generate data regarding the wear element.

In further embodiments, the invention encompasses an integrated micro-electromechanical (MEMS) spark-induced breakdown spectroscopy (SIBS) apparatus for detecting a wear element in a liquid is provided. In embodiments, the apparatus comprises a MEMS form factor, a voltage source, an apparatus for focusing a stream of the liquid into voltage source, and collection optics to direct light to the entrance slit of a spectrometer, and a spectrometer to measure the spectrum of plasma generated by the voltage source and generates data regarding the wear element.

In further embodiments, the invention encompasses methods for clearing collection optics of deposited oil film, such as, for example, vibration from small piezoelectric elements integrated in proximity to optical surfaces, directional air or compressed gas jets, or specialty oleophobic coatings.

In further embodiments, the invention encompasses elimination of all or most free space optics to allow integration and miniaturization. Methods include, for example, use of fiber-based excitation and/or collection optics, and an arrayed waveguide grating or other solid state diffractive element.

In further embodiments, the invention encompasses a system on a chip including an arrayed waveguide grating with single element detectors tuned to particular frequencies (lines) of interest for the analysis of wear metals in oil, for purposes of monitoring those concentrations with a spectrometer of minimum size and maximum simplicity to maximize integration and minimize size.

In further embodiments, the invention encompasses a laser spark to create a spark, and then inject higher levels of charge through the use of an external current (charge) source circuit. In this manner, larger and more energetic plasma can be created, while maintaining the sharp temporal and spatial focus of the laser to initiate the spark.

Further embodiments, features, and advantages of the embodiments, as well as the structure and operation of the various embodiments, are described in detail below with reference to accompanying drawings.

### BRIEF DESCRIPTION OF THE FIGURES

FIG. 1 is an illustration of the periodic table that indicates the approximate sensitivity for the various elements and includes an indication of the relative detection limits for the elements using LIBS.

FIG. 2 shows an exemplary embodiment of a MEMS LIBS apparatus as described herein.

FIG. 3 shows an integrated optical/microfluidics apparatus as described herein.

FIG. 4 shows a plunger interface with integrated filter actuator as described herein.

FIG. 5 shows a microdroplet ejection apparatus in accordance with embodiments described herein.

FIG. 6 shows a focused jet approach as described in embodiments herein.

FIG. 7 shows a flowchart of a LIBS operation.

FIG. 8 shows oil sample mounting for analysis.

FIG. 9 shows data collected from two oil samples absorbed into a graphite matrix.

FIG. 10 shows a comparison of two oil samples.

FIG. 11 shows an overlay of Sample #1 analysis with variations in analyzer delay.

FIG. 12 shows an overlay of Samples #1 and #2 through Pt aperture.

FIG. 13 shows an additional overlay of Samples #1 and #2 through Pt aperture.

FIG. 14 shows an additional overlay of Samples #1 and #2 through Pt aperture.

FIG. 15 shows an additional overlay of Samples #1 and #2 through Pt aperture.

FIG. 16 shows an additional overlay of Samples #1 and #2 through Pt aperture.

FIG. 17 shows a method for creating and controlling an oil/air interface.

FIG. 18 shows a closed loop regulator system described herein.

FIG. 19 shows a sinusoidally-driven MEMS diaphragm for varying meniscus height as described herein.

FIG. 20 illustrates a standard prism and the function of a selective arrayed waveguide spectrometer of the invention. The AWG may be designed to pass a broad band of light for each of the final output waveguides, thus separating the initial spectrum into smaller pieces.

FIG. 21 illustrates a laser source design including a ring resonator design.

FIG. 22 illustrates an exemplary device for discharge induced breakdown spectroscopy.

FIG. 23a illustrates a graphite electrode, which produces an excellent signal-to-noise ratio (SNR) during spark discharge. FIG. 23b, illustrates a series of 10,000 sparks in 1000 spark increments showing gradual erosion of anode. In illustrative non-limiting embodiments, even after about 10,000 sparks the electrode is still producing a reliable spark and spectrum.

FIG. 24 illustrates an illustrative embodiment of an integrated step up supply to provide high voltage, which allows for a continuous potential and a small footprint.

FIG. 25 illustrates an illustrative embodiment of a flashlamp capacitor charger to increase power up to ˜300V through low power transformer and discharge capacitor through HV power transformer to create spark. FIG. 25 illustrates an illustrative embodiment of this design.

FIG. 26 illustrates a non-limiting ignition triggered capacitive probe of the invention.

FIG. 27 illustrates an exemplary Czerny-Turner (CT) spectrometer, which includes a spectrometer including a slit, collimator, dispersive element (grating or prism), focusing mirror and a detector array.

FIG. 28 illustrates an exemplary prototype of the invention.

FIG. 29 illustrates a schematic of the process of the invention.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. It should be understood that use of the term “about” also includes the specifically recited amount.

Technical and scientific terms used herein have the meaning commonly understood by one of skill in the art to which the present application pertains, unless otherwise defined. Reference is made herein to various methodologies and materials known to those of skill in the art.

The apparatuses of the invention can be utilized in any system that requires a lubricating oil, such as an automobile, train, marine, aircraft, industrial device, compressor, and wind turbine. The term “automobile” refers to, for example, a passenger car, passenger truck, as well as large, cargo trucks, tow trucks, etc. The term “marine” refers to, for example, a ship or a boat. The term “industrial device” refers to, for example, machinery used in an industrial or commercial setting that requires lubricating oil.

The invention generally encompasses an integrated micro-electromechanical (MEMS) breakdown spectroscopy apparatus for detecting a wear element in a liquid, the apparatus comprising:

a MEMS substrate form factor;

a breakdown means integrated with the MEMS substrate form factor;

means to generate a plasma;

a spectrometer configured to measure a spectrum of light emitted by the plasma and produce data regarding the wear element; and

electronics for transmitting the data regarding the wear element.

In certain embodiments, the breakdown means is laser induced breakdown spectroscopy.

In certain embodiments, the breakdown means is spark induced breakdown spectroscopy.

In certain embodiments, the spectrometer is a selective arrayed waveguide spectrometer.

In certain embodiments, the spectrometer is a Czerny-Turner (CT) spectrometer.

In certain embodiments, the liquid is an oil-based lubricant.

In certain embodiments, the laser is an IR laser.

In certain embodiments, the laser is a sub-nanosecond pulse laser.

In certain embodiments, the form factor is between about 30 cm³ and about 100 cm³.

In certain embodiments, the oil-based lubricant is selected from the group consisting of:

a. an automotive lubricant;

b. a marine lubricant;

c. an aircraft lubricant;

d. an industrial device lubricant;

e. a compressor lubricant; and

f. a wind turbine lubricant.

In certain embodiments, the wear element is selected from the group consisting of:

a. Na;

b. Mg;

c. Al;

d. Si;

e. Mn;

f. Fe;

g. Ni;

h. Cu;

i. Zn; and

j. Mo.

In certain embodiments, the wear element in the liquid is detected at a level of between 0.1 and 200 parts per million.

In certain embodiments, the MEMS apparatus further comprises a receiver unit remotely located from the MEMS form factor.

In certain embodiments, the MEMS apparatus is incorporated in a machine, for example, a car, a truck, a boat, a ship, an aircraft, an industrial machine, a compressor, and a wind turbine.

The invention further comprises a method of detecting wear elements in a liquid, comprising:

providing a liquid sample;

contacting the liquid sample with a means to generate a plasma; and

detecting one or more wear elements in the plasma with laser-induced breakdown spectroscopy,

wherein the liquid is contacted with a laser that is integrated into a MEMS form factor.

In certain embodiments, the liquid is an oil-based lubricant.

In certain embodiments, the one or more wear elements are wear metals.

In certain embodiments, the invention comprises transmitting data regarding the one or more wear elements to a receiver remotely located from the MEMS form factor.

The invention further encompasses an integrated micro-electromechanical (MEMS) spark-induced breakdown spectroscopy (SIBS) apparatus for detecting a wear element in a liquid, the apparatus comprising:

a MEMS substrate form factor;

high voltage source connected to electrodes incorporated with the MEMS substrate form factor;

one or more focusing optics or reflectors;

a microfluidic flow channel comprising the liquid;

collection optics to gather light emitted by the liquid generated by the spark; and

a spectrometer configured to measure the spectrum of the light emitted by the plasma generated by the laser and generate data regarding the wear element.

In certain embodiments, the liquid is an oil-based lubricant.

In certain embodiments, the oil-based lubricant is selected from the group consisting of:

a. an automotive lubricant;

b. a marine lubricant;

c. an aircraft lubricant;

d. an industrial device lubricant;

e. a compressor lubricant; and

f. a wind turbine lubricant.

In certain embodiments, the wear element is selected from the group consisting of:

a. Na;

b. Mg;

c. Al;

d. Si;

e. Mn;

f. Fe;

g. Ni;

h. Cu;

i. Zn; and

j. Mo.

The invention generally encompasses an integrated micro-electromechanical (MEMS) apparatus sensor (e.g., laser-induced breakdown spectroscopy (LIBS) or spark-induced breakdown spectroscopy (SIBS)) incorporating a selective arrayed waveguide spectrometer (SAWS) for detecting a wear element in a liquid, the apparatus comprising:

a MEMS substrate form factor;

optionally a breakdown means (for example a laser or spark) integrated with the MEMS substrate form factor;

an optical fiber or freespace optical elements configured to transmit a pulse from the breakdown means to the liquid sample and to generate a plasma;

a spectrometer for example a SAWS spectrometer configured to measure a spectrum of light emitted by the plasma and produce data regarding the wear element; and

electronics for transmitting the data regarding the wear element.

In certain embodiments, the liquid is an oil-based lubricant.

In certain embodiments, the breakdown means is a laser such as an IR laser.

In certain embodiments, the laser is a sub-nanosecond pulse laser.

In certain embodiments, the form factor is between about 30 cm³ and about 100 cm³.

In certain embodiments, the oil-based lubricant is selected from the group consisting of: a. an automotive lubricant; b. a marine lubricant; c. an aircraft lubricant; d. an industrial device lubricant; e. a compressor lubricant; and f. a wind turbine lubricant.

In certain embodiments, the wear element is selected from the group consisting of Na; Mg; Al; Si; Mn; Fe; Ni; Cu; Zn; and Mo.

In certain embodiments, the wear element in the liquid is detected at a level of between 0.1 and 200 parts per million.

In certain embodiments, the MEMS apparatus further comprises a receiver unit remotely located from the MEMS form factor.

In certain embodiments, the invention encompasses a machine comprising the MEMS apparatus. In certain embodiments, the machine is selected from the group consisting of a car, a truck, a boat, a ship, an aircraft, an industrial machine, a compressor, and a wind turbine.

Another embodiment encompasses method of detecting wear elements in a liquid, comprising:

providing a liquid sample;

contacting the liquid sample with a source (e.g., a laser) to generate a plasma; and

detecting one or more wear elements in the plasma with laser-induced breakdown spectroscopy,

wherein the liquid is contacted with a laser that is integrated into a MEMS form factor.

In certain embodiments, the liquid is an oil-based lubricant.

In certain embodiments, the one or more wear elements are wear metals.

In certain embodiments, the invention further comprises transmitting data regarding the one or more wear elements to a receiver remotely located from the MEMS form factor.

In another embodiment, the invention encompasses an integrated micro-electromechanical (MEMS) apparatus (including LIBS, SIBA, or SAWS) for detecting a wear element in a liquid, the apparatus comprising:

a MEMS substrate form factor;

an integrated breakdown source with the MEMS substrate form factor;

one or more focusing optics or reflectors;

a microfluidic flow channel comprising the liquid;

collection optics to gather light emitted (e.g., by a plasma generated by the laser); and

a spectrometer, for example, configured to measure a spectrum of the light emitted by plasma generated by a laser and generate data regarding the wear element.

In another embodiment, the invention encompasses an integrated micro-electromechanical (MEMS) apparatus for detecting a wear element in a liquid, the apparatus comprising:

a porous filter;

a breakdown source in contact with the porous filter;

a plunger for drawing a liquid into the porous filter;

collection optics to gather light emitted by the plasma generated by the laser; and

a spectrometer configured to measure the spectrum of the light emitted by the plasma generated by the laser and generate data regarding the wear element.

An integrated micro-electromechanical (MEMS) laser-induced breakdown spectroscopy (LIBS) apparatus for detecting a wear element in a liquid, the apparatus comprising:

a MEMS form factor;

a laser;

an apparatus for ejecting a droplet of the liquid into a focal point of the laser;

collection optics to gather light emitted by the plasma generated by the laser; and

a spectrometer configured to measure the spectrum of the light emitted by the plasma generated by the laser and generate data regarding the wear element.

In certain embodiments, the liquid is an oil-based lubricant.

In certain embodiments, the breakdown means is a laser such as an IR laser.

In certain embodiments, the laser is a sub-nanosecond pulse laser.

In certain embodiments, the form factor is between about 30 cm³ and about 100 cm³.

In certain embodiments, the oil-based lubricant is selected from the group consisting of: a. an automotive lubricant; b. a marine lubricant; c. an aircraft lubricant; d. an industrial device lubricant; e. a compressor lubricant; and f. a wind turbine lubricant.

In certain embodiments, the wear element is selected from the group consisting of Na; Mg; Al; Si; Mn; Fe; Ni; Cu; Zn; and Mo.

In certain embodiments, the wear element in the liquid is detected at a level of between 0.1 and 200 parts per million.

In certain embodiments, the MEMS apparatus further comprises a receiver unit remotely located from the MEMS form factor.

An integrated micro-electromechanical (MEMS) laser-induced breakdown spectroscopy (LIBS) apparatus for detecting a wear element in a liquid, the apparatus comprising:

a MEMS form factor;

a laser;

an apparatus for focusing a stream of the liquid into a focal point of the laser;

collection optics to gather light emitted by the plasma generated by the laser; and

a spectrometer configured to measure the spectrum of the light emitted by the plasma generated by the laser and generate data regarding the wear element.

In certain embodiments, the liquid is an oil-based lubricant.

In certain embodiments, the breakdown means is a laser such as an IR laser.

In certain embodiments, the laser is a sub-nanosecond pulse laser.

In certain embodiments, the form factor is between about 30 cm³ and about 100 cm³.

In certain embodiments, the oil-based lubricant is selected from the group consisting of: a. an automotive lubricant; b. a marine lubricant; c. an aircraft lubricant; d. an industrial device lubricant; e. a compressor lubricant; and f. a wind turbine lubricant.

In certain embodiments, the wear element is selected from the group consisting of Na; Mg; Al; Si; Mn; Fe; Ni; Cu; Zn; and Mo.

In certain embodiments, the wear element in the liquid is detected at a level of between 0.1 and 200 parts per million.

In certain embodiments, the MEMS apparatus further comprises a receiver unit remotely located from the MEMS form factor.

### Wear Element Detection

Wear element is used herein to mean an element of the period table that is the product of mechanical wear or other deterioration or use of a liquid, such as a lubricating fluid in an engine. Concentrations of wear elements can be used to determine whether a system is approaching failure, allowing further damage to be avoided. Specific applications of lubricant analysis include the detection of cylinder damage in reciprocating engines, indicating worn or broken anti-friction bearings and retainers, and the misalignment of gears, which would lead to scoring. All such progressive failures add to the wear element (including wear metal) content of liquids, include lubricating fluids (e.g., oil) and hydraulic fluid systems.

Traces of wear elements in used lubricants generally differ in origin and their concentrations provide important information on the source and extent of the deterioration. Iron is the most common element; the wear of cylinder walls, valve guides, piston rings, bearings, and spring gears all contribute to elevated Fe levels. In machinery, copper is usually present in the form of alloys such as bronze or brass. Common sources of Cu include rod bearings, oil coolers, gears, valves, turbocharger bushings, and radiators. Aluminum appears during fatigue of spacers, washers, pistons, and crankcase of reciprocating engines and also from bearing cages in planetary gears. Magnesium typically arises from the wear of component housings. Sodium can arise from coolant leaks and is also present in grease. Zinc often arise from the wear of brass components, but can also be present in neoprene seals and greases. Other elements such as Ba, Ca, Mn, and Mo can be present in oil as additives.

### Laser Induced Breakdown Spectroscopy (LIBS)

Laser induced breakdown spectroscopy (LIBS) is an application of optical emission spectroscopy that employs the use of an ablation laser to remove material from a surface (including a liquid surface). Plasma formed from the vapor plume of ablated material generates the optical emission spectrum. Various detectors are available to analyze the emission spectra that can provide broadband detection for the elements present in the ablated region or high sensitivity for low levels of elemental contribution. Detection sensitivities can be achieved in the ppb to low ppm range for most elements and, excluding the noble gases, most elements from hydrogen through astatine can be studied effectively.

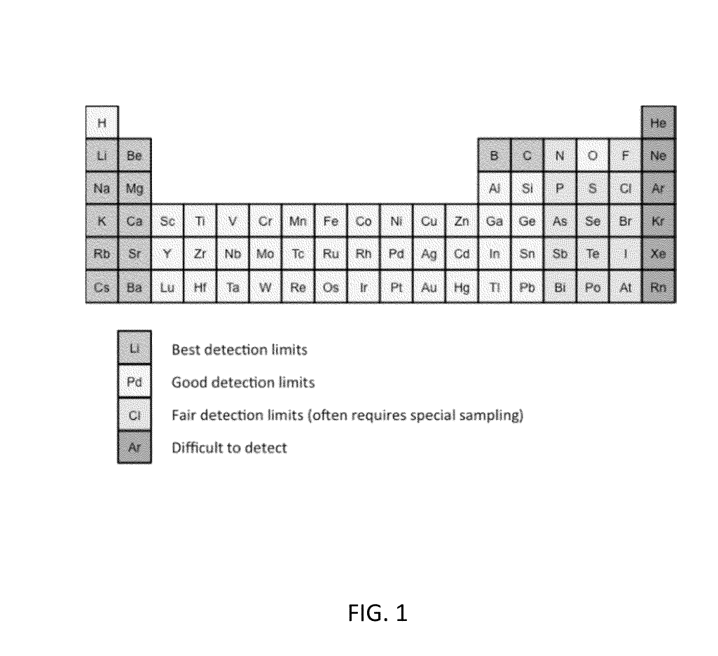

FIG. 1 is an illustration of the periodic table that indicates the approximate sensitivity of LIBS for the various elements and includes an indication of the relative detection limits for the elements using an apparatus described herein. The variable that has the greatest effect on the detection of various elements is the delay time before signal collection. Different elements have specific time regimes in the plasma burn where they are more likely to provide a signal. Tuning the detection for the specific element/plasma correlation optimizes the detection level for specific elements. The number of elements of interest in this study includes those, which have differing delay time optima, requiring that the measurements be made successively to obtain data for all elements.

In LIBS, a short intense laser pulse is used to ionize a small area of a liquid. The hot dense plasma created by the laser pulse expands into the ambient gas and cools rapidly during the initial expansion. As the plasma cools, electrons and ions recombine, and then decay from higher to lower energy states, emitting electromagnetic radiation and wavelengths characteristic of the elemental composition of the original liquid, i.e., an oil based lubricant. The key advantages of LIBS are: (a) Measurement speed: A LIBS measurement can be made in well under a millisecond. (b) Universal sensitivity: LIBS can detect all conventional elements in solids, liquids or gaseous form. (c) Limited sample preparation: Little or no sample preparation is required. Because such a small amount of material is consumed during the LIBS process the technique is considered essentially non-destructive or minimally destructive, and with an average power density of less than one watt radiated onto the specimen there is almost no specimen heating surrounding the ablation area, (d) Environmentally robust: LIBS can be performed under an extremely broad range of conditions. (e) Small Size: LIBS systems can utilize microchip lasers and detectors and can be reduced in size to perform microanalysis on emerging Lab-on-a-Chip form factors.

LIBS systems generally contain a load-locked sample introduction compartment, through which a specially-prepared planar target containing the sample is introduced. The target is then brought into careful mechanical alignment with a laser beam that is brought to a tight focus on the target's surface. The high energy density of the focused and pulsed laser causes a portion of the sample to be turned into a plasma (a “spark”), which emits light as excited electrons decay back down to their ground state.

Suitable levels of detection (LOD) delivered by the apparatus described herein meet/exceed a requirement of <200 ppm (suitably between 0.1 and 200 ppm). Precision is often better than 5%. Representative LOD values for wear elements in ppm are:

Na 8-24 ppm

Mg 0.4-1.8 ppm

Al 15-35 ppm

Si 14-45 ppm

Mn 6-20 ppm

Fe 11-20 ppm

Ni 34-47 ppm

Cu 6.1-2.4 ppm

Zn 11-11.4 ppm

Mo 27-31 ppm

Typical applications using conventional LIBS techniques utilize laser pulse energies in the range of 10-100 mJ and their typical laser focal spot size are on the order of hundreds of microns.

Utilizing a Micro-Electro-Mechanical System (MEMS) or Lab-on-a Chip form factor, laser pulse energies down to the hundreds of micro joules to create the LIBS plasma are suitably used while still realizing sensitivities comparable to conventional LIBS techniques.

Some characteristics of apparatus described herein:

Double pulse excitation has shown to improve resolution from 6 to 40 times when compared to single pulse.

Use of sub-nanosecond pulse lasers results in a shorter pulse leading to higher precision and repeatability.

In embodiments, the apparatuses of the invention can operate 24 hours, 7 days a week on the same duty cycle as the equipment it is installed on. In embodiments, the analysis of the wear elements is continuous and in real time. In additional embodiments, the wear elements in the lubricant are repeatedly sampled to obtain a statistical sampling over an extended period of time. The processor associated with the apparatus can then discern and report the differential changes in each of the wear elements detected. The repeatability precision is between 1 and 20%, preferably between 2 and 15%, preferably between 3 and 10%, and preferably between 4 and 8%, and preferably 5%.

The present invention provides apparatuses and methods that provide an accurate understanding of a liquid, such as a lubricating fluid (e.g., oil), which gives insight into the true operating status and condition of the liquid. In embodiments, an integrated system is provided for continuous monitoring of multiple properties of a liquid derived from measurements from a plurality of sensor modalities within a liquid-based closed-system environment. Suitable embodiments utilize a combination of advanced Micro-Electro-Mechanical Systems (MEMS) and semiconductor techniques to place the laboratory tests into close proximity with the liquid flow to continuously and concurrently analyze the fluid and report these parameters individually to a programmable computer to provide parallel and integrated real-time analysis of the liquid condition.

It is important to set thresholds for detection of foreign contaminants in the oil. For example, a sufficient quantity of water over time can cause corrosion of critical elements normally protected by the lubricating fluid. Based on these thresholds, certain alerts and notices can be provided, either transmitted through an output interface or polled by a wireless interface, optionally using a portable hand-held device, such as a smart phone. To validate the ongoing assessment of the liquid condition, a secondary check can be done to verify the measurements through periodic laboratory sampling. External validation can be part of the conforming calibration process during initial testing of the MEMS LIBS system. External validation can also qualify additional lubricating fluids and operating environments. Once the baseline is understood, the thresholds across all the integrated measurements can be programmed into the semiconductor to provide the alerting functionality over and beyond the integrated measurement data outputs.

In additional embodiments, the systems and methods described herein detect use of the wrong fluid or unsuitable lubricating fluid that may be mistakenly introduced into the lubrication system. Operating machinery with the wrong lubricating fluid can cause irreparable harm if not immediately remediated.

A control system integrates disparate sensors, utilizing patterns of sensor conditions to “recognize” or “diagnose” sets of conditions worthy of further attention. Established mathematical algorithms for such analysis include and are not limited to Kalman filtering (and enhanced Kalman filtering), hidden-Markov models, Bayesian analysis, artificial neural networks or fuzzy logic. These control systems can be implemented readily in software, firmware or hardware, or a combination thereof. (See: “Solutions for MEMS Sensor Fusion,” Esfandyari, J, De Nuccio, R, Xu, G., Solid State Technology, July 2011, p. 18-21; the disclosure of which is incorporated by reference herein in its entirety.)

In further embodiments, additional understanding of the fluid properties under different machinery operating conditions can be gained, for example, including “at rest” when the system is not operating, or at “peak heat,” which may actually occur after the system shutdown. Temperatures may increase after shutdown when no cooling fluid is circulating. Fluid properties will change as the fluid heats and cools. Measuring these changes across the short heating or cooling interval can yield valuable additional indications and insights into the properties of the lubricating fluid. Deviations may cause the control system to request measurements not only when the machinery is operating but also upon startup or shut down, for example.

The present application overcomes a number of limitations of traditional diagnostics. First, the traditional time delay from fluid sampling to testing may place critical equipment at risk of damage. Sometimes the lubricating fluid is sampled at the time it is being exchanged. While potentially useful for providing insight into the wear of internal parts, machinery may be operated in a potentially unsafe condition until the results are returned from the laboratory. Second, the lubricating fluid may be exposed to extreme temperatures during operating transients, which can be often in excess of 150° C., potentially causing some breakdown of additives in the lubricating fluid. Such problems are not usually detected, as the equipment often is “turned off” during these conditions. Although there is no new heat being generated, residual heat is transferred into the lubricating fluid and can potentially impact its performance. Such temperature extremes often require special engineering effort to design integrated in-situ sensing systems to support reliable operation (e.g. from −50° C. to +150° C.). Further, sensors and other electrically active elements need to support this environment. Equally important is the support of various pressures that the lubricating fluid may experience during normal and high-load operations. An in-situ sensor framework must be designed to withstand the peak temperatures and pressures experienced within the lubrication system over time.

The MEMS LIBS apparatuses provided herein are suitably designed to withstand high temperatures of the engine lubricant. The optical measuring methods are based on proven high-temperature designs. The optical spectrum suitably ranges from UV to mid-IR in which the lubricating fluid is not emitting energy at high temperature, depending on the fluid and the environment and potential contaminants. The transmission range is measured in millimeters and the distance between the emitting element and the receiving element is precisely controlled using known MEMS manufacturing techniques. This distance between the optical emitting and receiving elements must be very accurate. All of these elements have been implemented and operate individually within these extreme temperature and pressure environment in such a manner as to relay useful data.

In embodiments, the apparatuses, systems and methods described throughout provide real-time monitoring of liquids such as those associated with high-temperature environments present within or associated with internal combustion engines (i.e., monitoring the fluid during engine activity without the delay of removing a sample). Suitably, the apparatuses, systems and methods monitor oil-based fluid lubricants normally used with internal combustion engines, as well as other liquids such as transmission fluids or glycol-based coolants such as anti-freeze, and other liquids in manufacturing environments and critical life-saving medical equipment used in the healthcare industry. Another aspect addressed is monitoring fluid with a sensor module that is continually submerged within the lubrication fluid. Another aspect addressed is the parallel and integrated real-time analysis of the liquid condition. This invention also addresses high temperatures and other conditions experienced in the operating environment of such machinery.

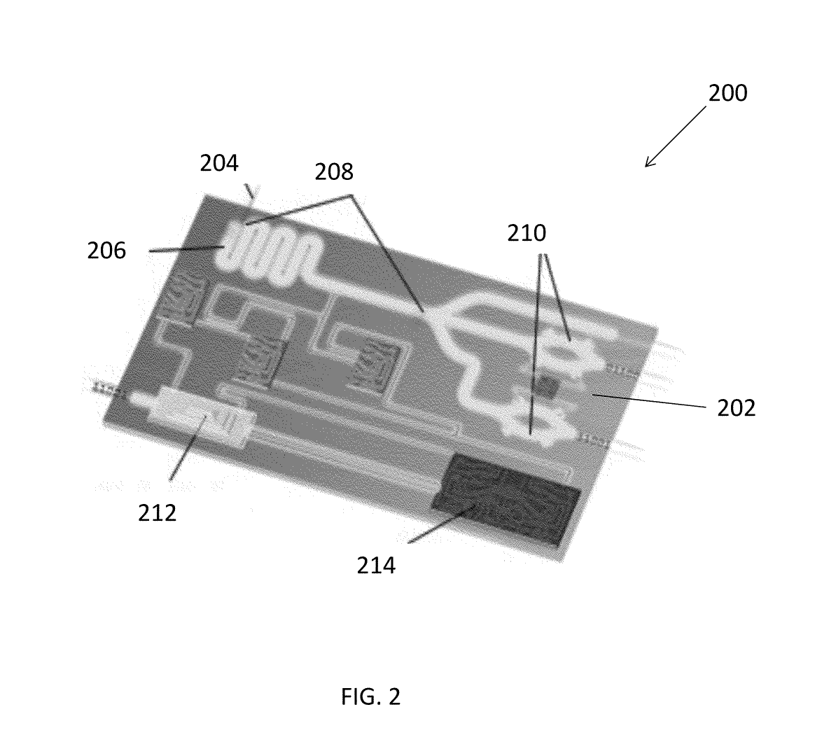

FIG. 2 shows an exemplary embodiment of a MEMS LIBS apparatus 200 as described herein. The terms “apparatus” “apparatuses” “device” and “devices are used interchangeably herein.

Suitably, the apparatuses provided herein include a MEMS substrate 202 form factor onto which the various components of the LIBS system are integrated. The LIBS system suitably comprises an optical fiber or freespace optical element (e.g., a lens) 204 for placement in proximity to a liquid (i.e., oil or other lubricant), a laser 206 integrated with the MEMS substrate form factor, collection optics to gather light emitted by the plasma generated by the laser (e.g., various mirrors 208 and modulators 210), as well as a spectrometer 212 configured to measure the spectrum of the light emitted by the plasma generated by the laser and generate data regarding the wear element, and electronics 214 for transmitting the data regarding the wear element, to carry out the various methods described herein. The various components of apparatus 200 are integrated with the MEMS substrate form factor, i.e., attached or otherwise made part of the form factor to allow for mechanical stability.

Optical fiber or freespace optical element 204 suitably delivers a laser pulse from laser 206 onto a liquid sample, such as oil, to generate a plasma (a “spark”), which emits light as excited electrons decay back down to their ground state. This emission is suitably measured by spectrometer 212, to generate data regarding the liquid (i.e., the chemical make-up of the liquid, including the presence of various wear elements (e.g., metals)) prior to being analyzed with electronics and suitably transmitted to an external monitoring receiver or calculating device that is remote from the MEMS form factor. In such embodiments, a system is provided comprising the apparatuses described herein and further comprising a receiver unit remotely located from the MEMS form factor. It should be understood that apparatus 200 is provided only for illustrative purposes, and various configurations of the components of the MEMS LIBS apparatus can be utilized.

Suitably, the laser for use in the MEMS LIBS apparatus described herein is an IR laser which may or may not be frequency doubled or quadrupled, in some embodiments it is a Nd:YAG solid state laser (a neodymium-doped yttrium aluminum garnet solid state laser).

In exemplary embodiments, the MEMS form factor is on a size scale of about 10 cm³ to about 400 cm³, suitably about 20 cm³ to about 300 cm³, about 30 cm³ to about 100 cm³, about 40 cm³ to about 75 cm³, about 45 cm³ to about 65 cm³, or about 50 cm³.

Application of LIBS to the continuous monitoring of engine oil means that there must be a practical way to reliably gather and present a sample of the oil to the focused laser spot. The boundary between the liquid and the ambient (air) provides the surface for the plasma spark, a necessary constraint since the plasma needs to develop into a highly compressible medium such as air, and will not form reliably in the bulk of a liquid. Using a stream boundary or a droplet surface approach satisfies this constraint; however as a practical matter the interface between the droplet or stream is constantly moving and changing. Thus, a stream or droplet must be generated in the field that is sufficiently well-controlled to meet the needed uniformity constraint. This is particularly true in the constantly moving and vibrating automotive environment. It is important to note in these considerations that it is the requirement for high energy density in the focused laser spot that makes it difficult to place the sample at the exact location of optimal laser focus. In short, the focal depth of the laser and the focal spot size are directly related: so the tighter the focus (smaller spot size) the smaller the focal depth (distance through which that spot is focused.)

One approach provided herein is to create a uniform flow of liquid (e.g., engine oil) through a fluidic channel fabricated on the MEMS device, which may be made of silicon, glass, plastic, metal, ceramic or some other substrate. At the point where the flowing oil is presented to the laser, a well-controlled air/oil interface is created and maintained. Several methods are described herein for exerting that control. This interface is maintained with a high degree of precision within the focal depth of the focused laser spot, so that the focused energy density is high enough with each laser pulse to produce a consistent and reliable spark. If the location of this interface changes by more than the laser focusing optics focal depth (often 10 microns or less) then the laser is sufficiently defocused by the time it reaches the oil/air interface to produce a non-uniform spark or to fail to ignite a spark entirely.

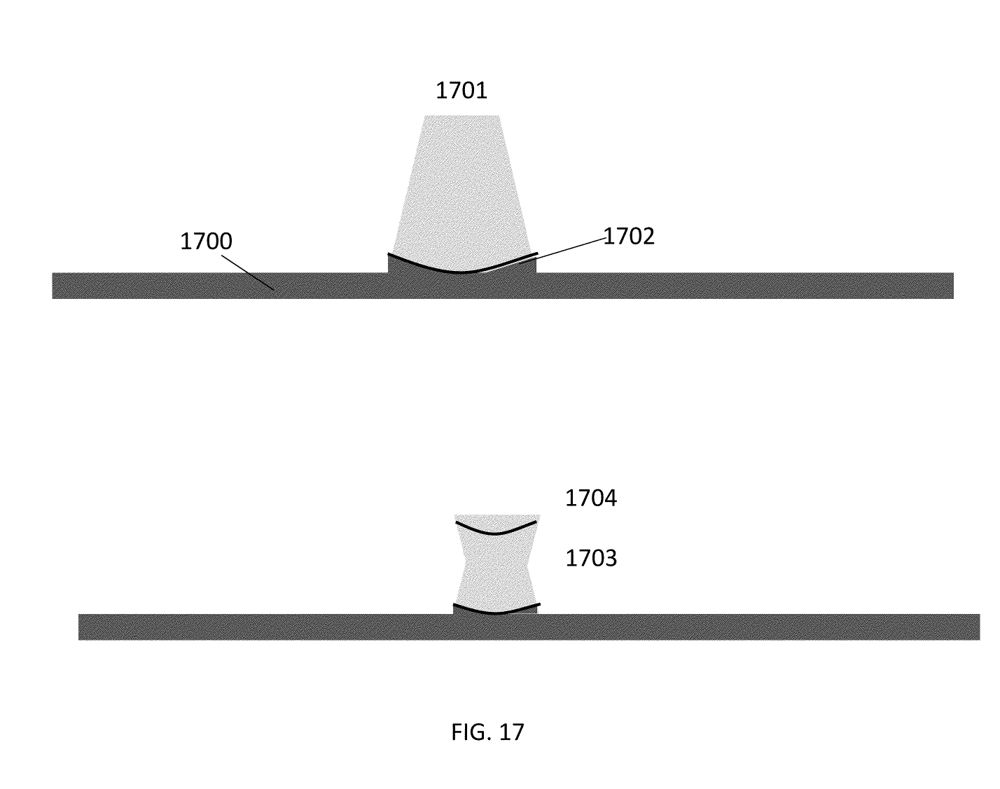

Described herein are multiple methods for creating and controlling the oil/air interface, for example a trapped volume of air can be created to apply a constant pressure on the oil/air meniscus. This is suitably turned into a closed loop system, in which pressure on the oil side is sampled and fed back to a diaphragm on the air side to match the pressure exactly across the meniscus. Alternatively, by varying the cross section of the reservoir of the trapped volume, a relatively high change in pressure (and accompanying change in relative volume, delta(V)/V) can be converted into a small change in meniscus height. This is accomplished by making the meniscus area 1702 of the oil 1700 large relative to the total volume of trapped air, for example using a tapered reservoir 1701 as shown in FIG. 17. In some embodiments this reservoir may be additionally tapered outwards (see 1703 of FIG. 17) to provide a high degree of hydraulic advantage for a diaphragm (1704) which is incorporated opposite the meniscus to control the pressure in the reservoir and thus control meniscus height.

A different approach is to intentionally vary the location of the air/oil interface so that it moves repeatedly through the point of optimal focus. By comparing spark intensity or the strength of a particular line as a function of that driving function, one can suitably determine the data gathered from the point of optimal focus, and disregard other data.

In embodiments, the following features are provided by the apparatuses and methods described herein:

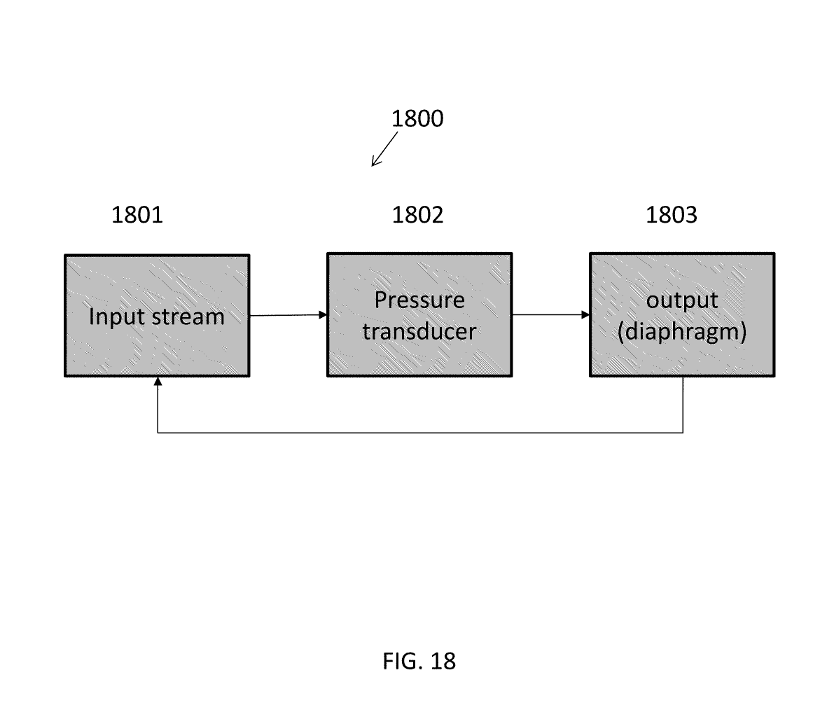

A highly compact MEMS pressure regulator for maintaining constant pressure in a trapped air volume; the regulator is a closed loop system (FIG. 18) for sampling input fluid stream pressure and adjusting it with an end effector (actuator, which in some embodiments is a diaphragm) to adjust the height of the meniscus to within desired limits, as show in FIG. 18. Closed loop system 1800 suitably comprises an input stream 1801, pressure transducer 1802 and diaphragm (output) 1803.

A micro-fabricated graduated ballast volume for regulating meniscus height; this embodiment is conceptually similar to that illustrated in FIG. 18 except that the air ballast volume illustrated therein is incorporated into the channel itself using the same microfabrication processing steps as used to define the channel itself.

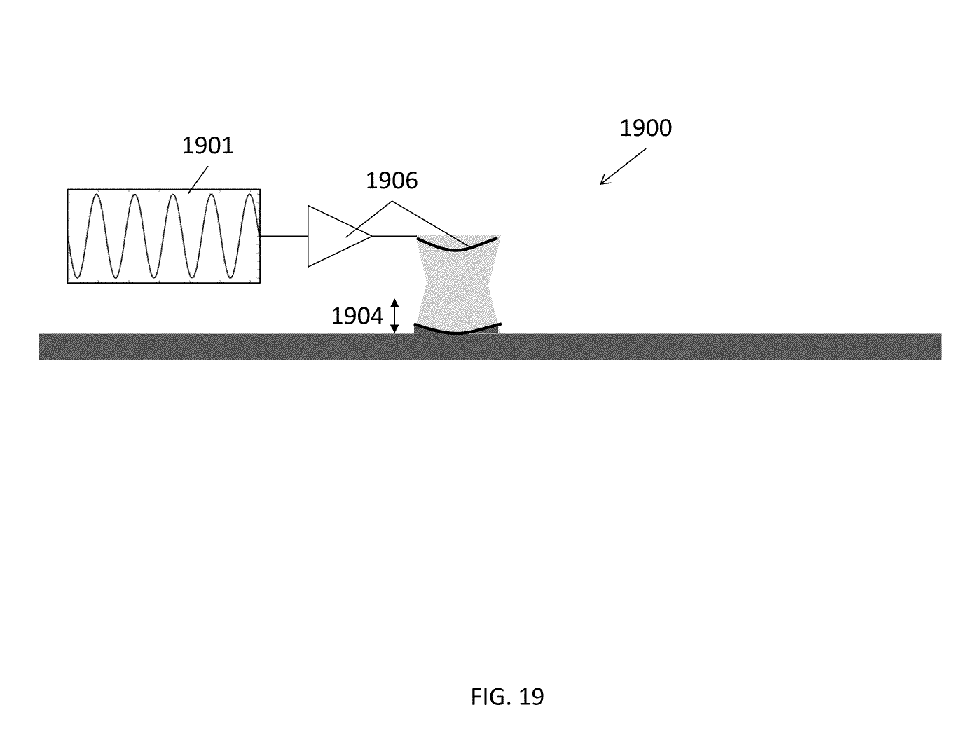

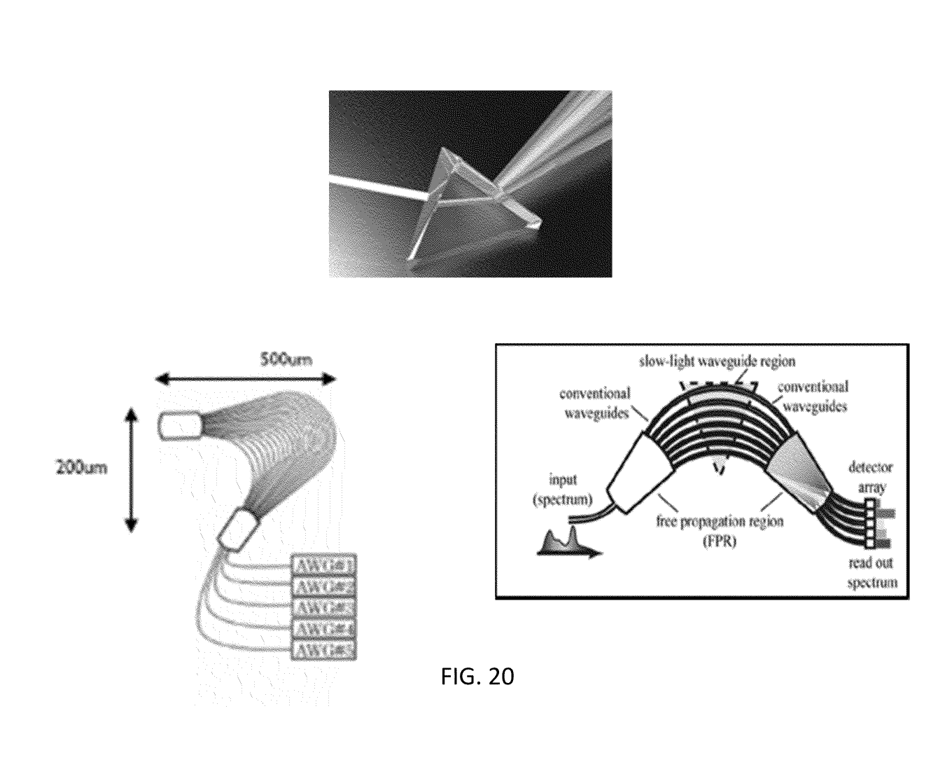

A sinusoidally-driven MEMS diaphragm (1900) for varying meniscus height 1904 can also be used. As show in FIG. 19, in this embodiment, a sinusoidal drive signal 1901 is applied to a driving diaphragm 1906, which results in a sinusoidal vertical motion of the meniscus height 1904, moving the oil/air interface through the focal point of the laser.

Methods for monitoring engine oil for the presence of wear elements using laser-induced breakdown spectroscopy are also provided herein. The methods and apparatuses described herein suitably allow for drawing and presenting a continuous sample of oil from a main oil circulation. The apparatuses provide many years of device lifetime without fouling or clogging, and can operate in extreme conditions of vibration, temperature and limited available space.

In certain embodiments, the laser source assembly comprises Monolithically Integrated Solid State Laser (MISSL). In certain embodiments, a monolithically integrated resonator provides the following advantages: eliminates tuning adjustments, thick film MEMS with coatings for mirrors; mechanical fiducials for rapid pick-and-place assembly; changes labor/materials cost relationship; convection cooled for low repetition rate, long recovery time removes active cooling; passive Q-switch reduces cost and control complexity; flashlamp pumped reduces cost and control complexity, simplifies assembly with no need to align pump to rod, and simplified pump chamber.

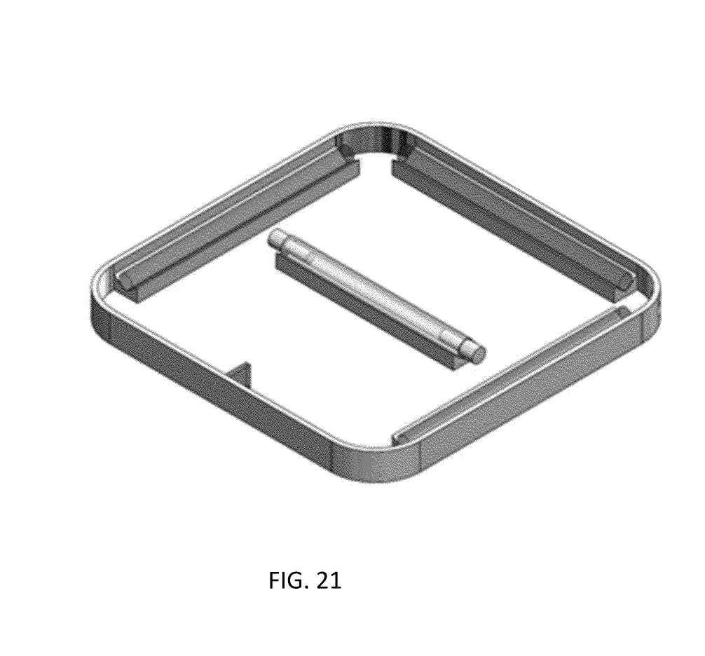

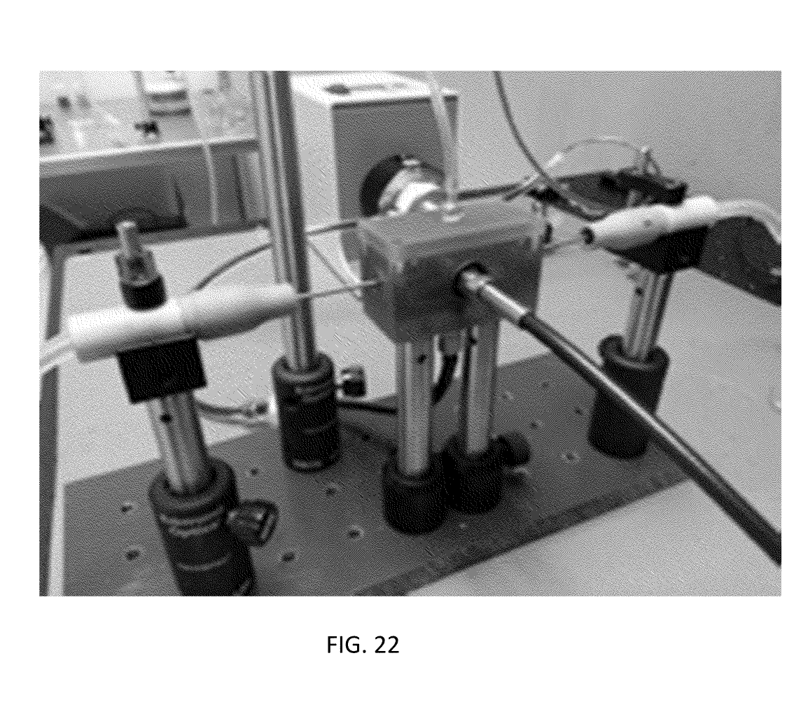

In certain embodiments, the laser source design includes a ring resonator design as illustrated in FIG. 21. This design supports three (3) gain rods; each at ⅓ the specified length; decreases size to ˜2.5 inches on a side; one arm for saturable absorber; micro-machined alignment grooves; pick-and-place assembly. In certain embodiments, the design also enables efficient use of space, with potential for Czerny-Turner spectrometer occupying same long path as the laser cavity (stacked above).

### Spark-Induced Breakdown Spectroscopy (SIBS) Sensor

The design, composition and uses of that system are explained above in the section regarding Laser-Induced Breakdown Spectroscopy. The rationale and basic approach described therein remains substantially the same; however, the spark generated during the high voltage breakdown of air (or other ambient) can replace the spark generated by a pulsed laser. This approach is referred to SIBS, for Spark Induced Breakdown Spectroscopy. Specifically, it became apparent in the course of building and testing prototype LIBS systems that a spark originating with voltage breakdown would offer benefits in terms of simplicity and robustness, as well as reduced component and assembly costs, as compared to a spark originating from a laser pulse.

As with LIBS, the purpose of the subject invention is to provide an autonomous and highly compact sensor platform for the real-time and continuous analysis of lubricating fluids. This analysis provides information not only about the state of the oil itself, but also about the state of the machinery or engine, enabling predictive maintenance. The current embodiment uses a voltage-induced breakdown spark to generate the spectral information. SIBS offers advantages in terms of reduced oil “splattering,” that is the introduction of sample material onto the collection optics, which can hinder proper functioning of the system. This improvement is attributed to the larger, hotter and longer spark produced by a spark discharge, as compared to that produced by laser ionization. In certain embodiments, this increased photon intensity allows viewing optics to be placed farther from the spark, thus ameliorating splattering problems. In other embodiments, the geometry of electrodes and the resulting discharge constrains the spark as compared to LIBS, thus also improving spatter. In certain embodiments, it may be possible to introduce intentional design features into the electrodes such that this spatial localization is optimized for minimization of splatter. As compared to commercially-available spark discharge systems for wear metal analysis in oil, SIBS has at least the following advantages: (1) autonomous and remote operation, with no operator intervention; (2) smaller form and fit, and much lower cost; (3) continuous, real-time analysis, rather than processing a single sample at a time under operator control, (4) easier alignment of collection optics to spark, via simple alignment to electrodes.

With SIBS, a current and voltage source is connected to a pair of closely-spaced electrodes. These electrodes can be composed of a variety of conductive or non-conductive materials including, but not limited to, graphite and other carbon-based materials, noble metals such as gold, platinum or iridium, other metals (titanium, steel), or ceramics. The sample to be analyzed is introduced between the electrodes, in the vicinity of the electrodes, or on one or both of the electrodes prior to sparking. A high voltage is applied between the electrodes using a voltage source, such that the breakdown voltage of air or other ambient gas is exceeded, causing ionization of the ambient, and the initiation of a low resistance pathway between the electrodes. A large amount of current is supplied to the electrodes, and flows with relatively little back voltage (and dissipated power) due to the drop in impedance between the electrodes. The resulting spark is composed of ionized and excited neutral atoms, including those drawn from the sample introduced prior to sparking. From here, the process of measuring elemental analysis is identical to that used in LIBS. As the excited neutrals comprising the sample decay down to the ground state, they emit radiation including characteristic spectral frequency lines, which are measured with a spectrometer and analyzed to determine the species present.

The SIBS system is composed generally of three subsystems: sample introduction, high voltage circuitry, and light collection. The sample introduction is a microfluidic chip or flow cell which is designed to introduce a small amount of oil, drawn from a circulating oil flow, into the sensor for analysis. This introduction may be carried out in a variety of ways including simple wetting of the electrode, a fine liquid jet, an aerosol spray or mist, or through the introduction of a droplet or small reservoir. In the preferred embodiment, the sample is introduced in such a manner as to wet the surface of the anode; this may be accomplished by injection through an aligned orifice or tube, or in an embodiment in which the anode is hollow and oil flows up the anode through an orifice at the top, allowing direct sample introduction. The high voltage system consists of a high voltage (20-40 kV) source that is also capable of delivering high currents (1-100 amps) in very short pulses. This source is connected to a pair of electrodes (anode and the cathode.) In some embodiments, the sample itself may form one of the two electrodes in this pair. Finally, the light collection system is composed of collection optics and an optical fiber that directs the light to the input of a spectrometer. The spectrometer is a dispersive device that separates the light into its constituent frequencies and measures the intensity in each of several frequency bins. The spectrometer may be any of a wide variety of designs including but not limited to Czerny-Turner or an arrayed waveguide grating.

In various embodiments, the invention enables low cost autonomous and continuous predictive maintenance for a variety of applications and platforms, including wind turbines, ocean-going vessels, mining equipment and automobiles. This system could also be used on other types of liquid samples, such as for drinking water and waste water monitoring, chemical and biological agent detection, characterization of crude oil and other materials, or for in-line monitoring of biological precursors, drugs or industrial agents. By dissolving a solid into liquid form it may also be possible to monitor solid phase samples as well.

Additional embodiments of this invention include: (1) methods for clearing collection optics of deposited oil film, such as: piezoelectric vibration, directional air or compressed gas jets; or specialty oleophobic coatings; (2) electrodes composed of chemical vapor deposited thin films of poly-diamond, carbon nanotubes, or other carbon material, or a vapor deposited or electroplated metal, which may be integrated into the same micro-fabricated substrate as the fluidic and/or electronics subsystems; (3) methods to extend the lifetime of electrodes, for example the use of specifically formulated ceramic or graphite materials, geometries that optimize lifetime, or the use of a variable (adjustable) gap or an array of electrodes; and (4) a high voltage source/delivery system that is piggy-backed on an automotive ignition system (“9th spark plug”).

In certain embodiments, the invention encompasses discharge induced breakdown spectroscopy (DIBS), which generates electrically induced plasma using a high voltage pulse generator preferably at atmosphere—breakdown voltage of air is ˜20 kV. In certain embodiments, the electrode material includes an electrode spectrum that does not interfere with oil spectrum. In other embodiments, the electrode is 99.995% pure electrode material with known constituents and minimal interferants. In certain embodiments, the method of introduction of oil is controlled to optimize its contact with the electrode, which provided excellent results, comparable to or better than, for example, LIBS.

TABLE 1

| | | | | | |
|---|---|---|---|---|---|
| | | | Electrical | | |
| Analysis | Electrode | Laser | Energy | Detection | |
| Technique | Material | Energy | Voltage | | Concentration |

Oil Jet Presentation

| | | | | | |
|---|---|---|---|---|---|
| LIBS | | 100 mJ | | 100% | 200 ppm |
| SIBS | Graphite | | 25 mJ | 100% | 1000 ppm |
| SIBS | Graphite | | 25 mJ | 100% | 200 ppm |
| SIBS | Gold | | 400 mJ | 0% | 1000 ppm |
| DIBS | Gold | 50 mJ | 5 kV | 50% | 1000 ppm |
| (LTS) | | | | | |
| DIBS | Gold | 25 mJ | 6.5 kV | 100% | 1000 ppm |
| (LCS) | | | | | |

Oil Aerosol Presentation (STP)

| | | | | | |
|---|---|---|---|---|---|
| LIBS | | 100 mJ | | 50% | 800 ppm |
| SIBS | Graphite | | 400 mJ | 75% | 1000 ppm |
| SIBS | Gold | | 400 mJ | 75% | 1000 ppm |
| DIBS | Gold | 100 mJ | 6.5 kV | 50% | 1000 ppm |
| (LTS) | | | | | |
| DIBS | Gold | 100 mJ | 6.5 kV | 50% | 1000 ppm |
| (LCS) | | | | | |

Other Presentation

| | | | | |
|---|---|---|---|---|
| LIBS | Graphite | 100 mJ | 100% | 200 ppm |
| LIBS | Graphite | 25 mJ | 100% | 1000 ppm |

LIBS Laser Induced Breakdown Spectroscopy
SIBS Spark Induced Breakdown Spectroscopy
DIBS “Dual” Induced Breakdown Spectroscopy
LTS Laser Triggered Spark
LCS Laser Continuous Spark

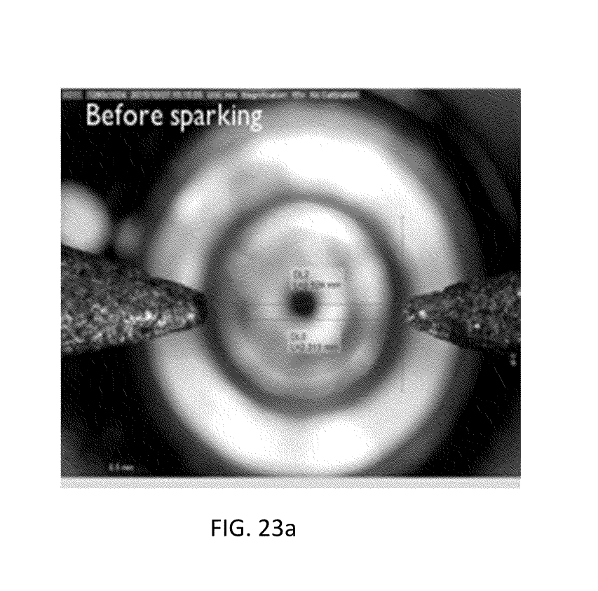

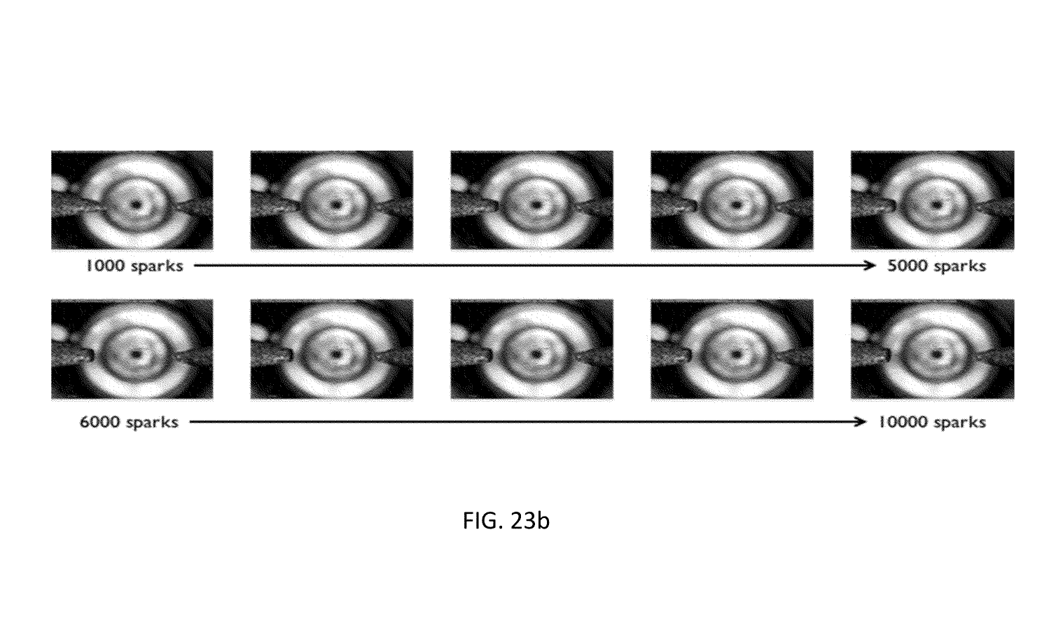

In an exemplary embodiment, a graphite electrode, illustrated in FIG. 23a, produces excellent signal-to-noise ratio (SNR) during spark discharge. In FIG. 23b, a series of 10,000 sparks in 1000 spark increments showing gradual erosion of anode. Even after about 10,000 sparks the electrode is still producing a reliable spark and spectrum.

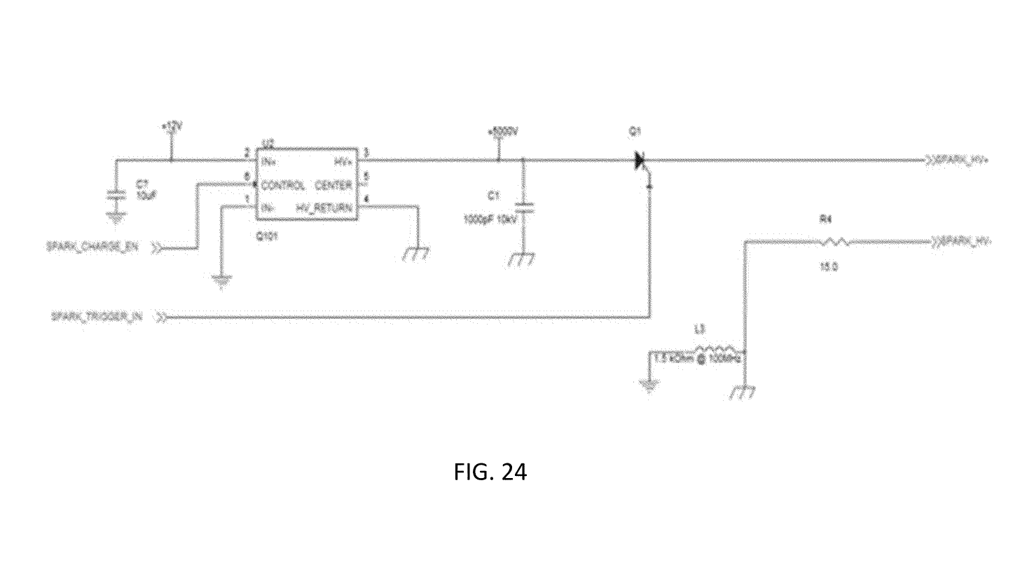

In certain illustrative embodiments, the invention encompasses an integrated step up supply to provide high voltage, which allows for a continuous potential and a small footprint. FIG. 24 illustrates an illustrative embodiment of this design.

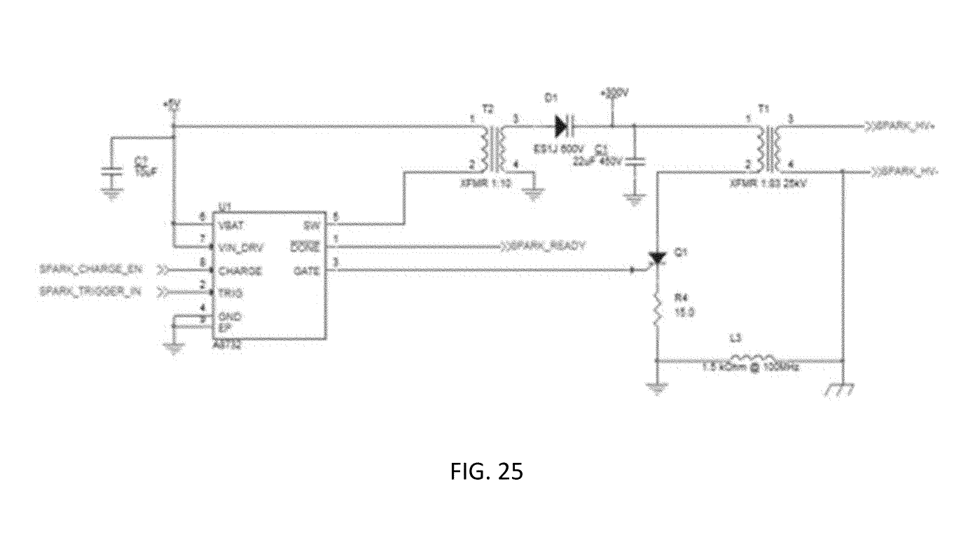

In certain illustrative embodiments, the invention encompasses a flashlamp capacitor charger to increase power up to ˜300V through low power transformer and discharge capacitor through HV power transformer to create spark. FIG. 25 illustrates an illustrative embodiment of this design.

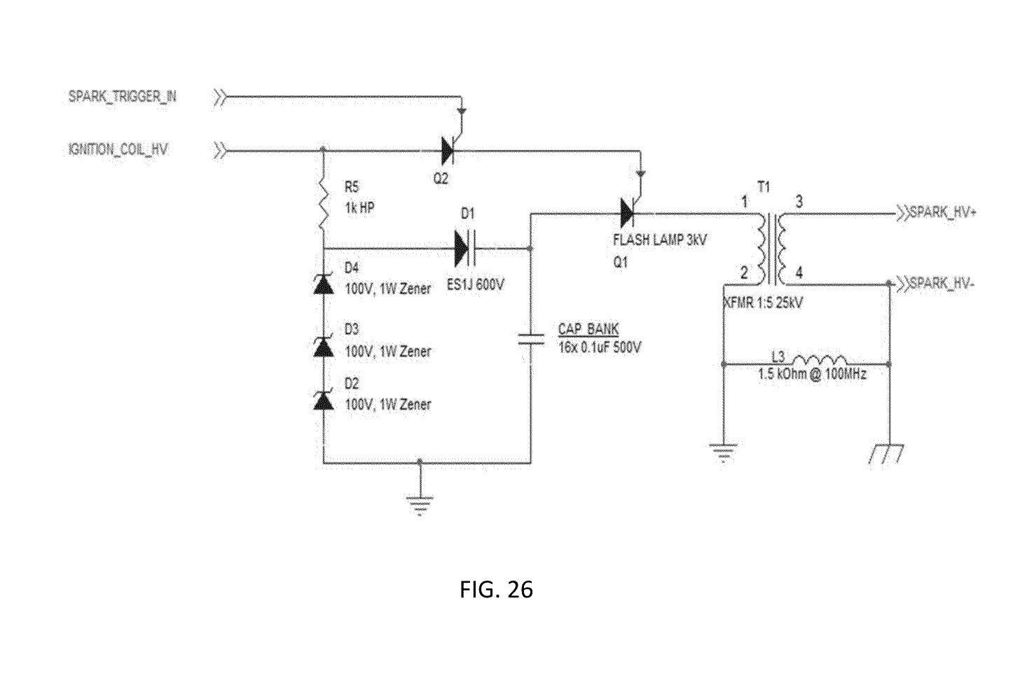

In certain illustrative embodiments, the invention encompasses an ignition triggered capacitive probe. In certain embodiments, the ignition triggered capacitive probe includes (1) a capacitor bank, a Zener diodes to charge, provides additional excitation energy; (2) Flashlamp, which normalizes voltage from ignition coil of initial spark and emission provides optical calibration, and (3) spark voltage provided by low-ratio step up transformer. FIG. 26 illustrates a non-limiting ignition triggered capacitive probe of the invention.

### Spectrometer Design: Selective Arrayed Waveguide Spectrometer (SAWS)

The traditional design of a spectrometer (or similarly a monochromator) results in a tradeoff between spectral range and resolution for the final device. This is a function of the resolution available in the detector array. Thus, a higher resolution device requires a narrower band of energy to be dispersed on a single element in the array. For a given detector array, this results in a reduction in the total range that the spectrometer may cover.

The detection of only specific wavelengths is preferred although they may require differing spectral widths around that band. These wavelengths are also not evenly spaced relative to one another. In traditional spectrometer design, the regions where there are no wavelengths of interest are wasted contributing to increased cost and size.

In certain embodiments, the invention encompasses a Wavelength Division Multiplexed (WDM) transmission system. These systems allow for the separation of multiple wavelengths carried in a single optical fiber into multiple fibers each carrying a separate wavelength. In certain embodiments, the invention includes an Arrayed Waveguide Grating (AWG) purpose. It had significant advantages over the optical power splitter configuration.

In certain embodiments, the AWG is configured with required center wavelengths and bandwidths while being low cost and small. Rather than being continuous, this device would be selective in what it detected.

In certain embodiments, the AWG is fabricated using simple planar optical waveguide technology, typically using Silicon on Insulator. Thus, it can be manufactured to high tolerances and with standard photolithographic techniques thereby driving the cost down. The optical waveguides at the output of the output slab waveguide determine the center of the passband and need not be evenly spaced. The spectral width of each passband is adjusted through additional parameters such as the slab waveguide size and the difference in path length between the waveguides. Additionally, the AWG can be easily cascaded such that the output of one AWG serves as the input to another. By using this technique, the desired characteristics of the spectrometer can be further customized for the specific application. Finally, single unit photodetectors can be easily placed at the final output waveguides.

Because of the arrayed design, the device does not suffer from the same dispersion to length ratio requirements of traditional spectrometers and can be made very small (˜1 cm2).

In certain embodiments, the invention encompasses an optical device consisting of passive optical structures fabricated to maintain waveguide characteristics for the frequencies/wavelengths of interest for the application.

In certain embodiments, the invention encompasses an input optical waveguide (fiber or planar) coupled to a slab waveguide. The input slab waveguide is responsible for distributing light to a number of conventional optical waveguides on the distal side of the slab.

In certain embodiments, the invention encompasses an array of traditional optical waveguides connecting the distal side of the input slab and the near side of the output slab. Said waveguides possess different path lengths incremented by a common length. For example, if the reference waveguide is 10, the second waveguide is 10+ l, the third is 10+2 l, etc. This sets a constant wavefront tilt, thus dispersion. The resolution of the resulting spectrometer will be a function of this wavefront tilt.

In the path of the traditional optical waveguides, additional elements may be introduced as long as they don't introduce any wavefront tilt distortion. For example, a waveplate may be inserted that compensates for any polarization mode dispersion or polarization dependent loss that could be encountered.

An output optical slab waveguide that is responsible for distributing light from each of the waveguides of the array to specific points on the distal end of the slab. The inner surface of the distal side of the slab waveguide can be thought to have the dispersion characteristics necessary that an array of photodetectors (e.g., CCD array) could be placed like in a traditional spectrometer. This could be one embodiment for future development, but it is not useful for the intended application. The CCD array would need to be placed precisely, thus incurring additional costs.

In the intended application, traditional output waveguides will be precisely placed at the distal end of the output slab waveguide in locations that correspond to the wavelength of interest for the spectrometer. These output waveguides would be an integral part of the design of the device's mask for photolithography. The key distinguishing characteristic of this invention is that the location of these waveguides would not be uniformly spaced as in the traditional method of using and manufacturing AWGs. They would be placed only where spectra of interest were located. This eliminates any unused areas of the spectrometer and optimizes the device.

Discrete photodetectors could be installed through automated means at the output of each of the output waveguides. These would not need high precision placement.

The AWG may be designed to pass a broad band of light for each of the final output waveguides, thus carving up the initial spectrum into smaller pieces. Rather than being fed to a photodetector, that signal would then be fed to an additional AWG for further division.

The device will possess a single optical waveguide input that may originate from the spectrographic system. The output of the device will be an electrical signal from each photodetector corresponding to the specified design for passband and center wavelength.

In certain embodiments, the spectrometer could be integrated with the excitation source for a complete, low cost application-specific analysis system.

In other embodiments, the invention includes a spectrometer including a slit, collimator, dispersive element (grating or prism), focusing mirror and a detector array. The dispersive element splits light into its constituent wavelengths. Design tradeoffs are available between the physical space available for dispersion, free spectral range, and resolution.

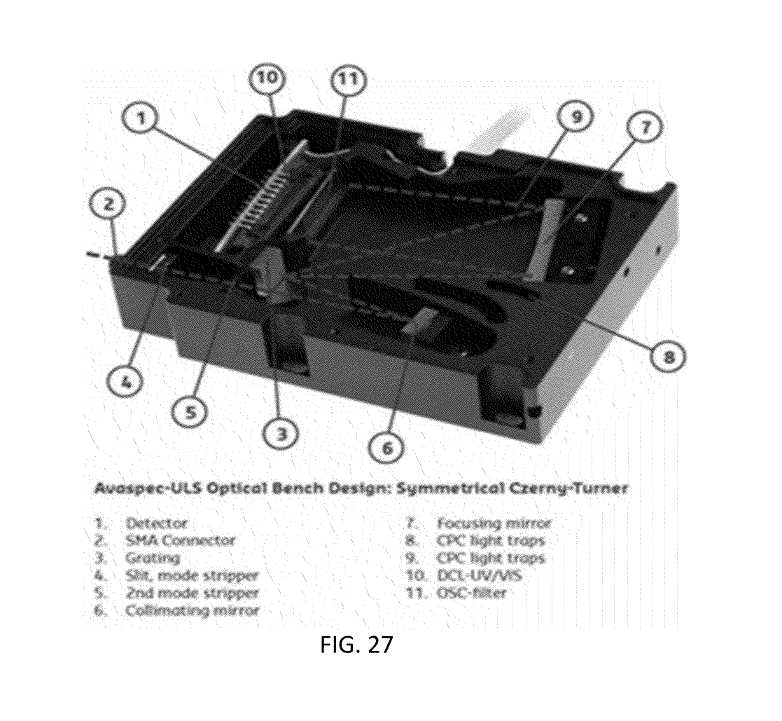

In certain embodiments, the spectrometer is a Czerny-Turner (CT) spectrometer design as illustrated in FIG. 27. In certain embodiments, the spectrometer allows for broad spectral range of 200-700 nm and high spectral resolution of about 0.1 nm. The spectrometer of the invention provides the benefits of high precision bulk optical components, critical alignment, high “touch time” for assembly, and wavelength calibration after assembly.

In certain embodiments, the Selective Arrayed Waveguide Grating (SAWG) is similar to that developed for wavelength division multiplexed (WDM) telecommunications systems. In certain embodiments, the spectrometer offers the following advantages, symmetric waveguide spacing supports standards (e.g., ITU G.694), configurable passbands and center wavelength, need not be symmetrical, building blocks are cascadable, loss is not proportional to channel count, design would be specific to analyte (i.e., Application Specific Spectrometer), fabricated using silicon on insulator techniques, single piece detectors placed on substrate, each detector signal represents specific band and specific wavelength, therefore specific emission line, additional detectors placed for normalization, noise reduction, calibration.

In certain embodiments, the SAW spectrometer is comprises the following:

(1) Arrayed Waveguide Gratings (AWG), for example, used in telecommunication sector as a low cost optical (de)multiplexe and operating principle similar to a phased array antenna

(selective wavelength interference);

(2) Composition includes

(i) Input slab

(ii) Channel waveguides, for example, the channel wave guides are designed to have a different path length each with a constant length increase (ΔL) causing a constant phase shift (wavefront tilt) at the exit

(iii) Output slab, for example, the output slab allows for constructive interference of the constituent wavelengths of light at the output waveguides and output waveguides connected to discrete photodetectors.

In certain, embodiments, the apparatus of the invention has the following advantages: (1) a planar device with a size no larger than, for example, 3 inches×2 inches; (2) no critical alignment or assembly of the components due to “monolithic construction;” (3) splatter contamination does not occur; (4) no contamination of optics; (5) simplifies fluidic design; (6) gating is not required, no continuum is generated; and (7) simplified electronics.

In certain embodiments, the spectrometer includes a spectral Range: 200 nm to 775 nm; a wavelength resolution: 0.1 nm; sensitivity: 310,000 counts/μW per ms integration time; size of less than 50 mm×50 mm×1.5 mm.

The Selective Arrayed Waveguide Spectrometer of the invention provides the following additional advantages: monolithic construction the devices allow complete fabrication on a single silicon wafer; a <0.2 nm resolution device with a 20 nm Free Spectral Range (FSR); a cascaded device with AWG's similar to the target device covers the 200-800 nm range; the FSR of each cascaded device is selected to cover the eight wavelength bins; there is low loss per split because each split has all of that particular wavelength component; and size is smaller than 40 mm×40 mm×0.5 mm.

In certain embodiments, SAWS fabrication to use conventional MEMS manufacturing process performed on a Silicon wafer (12″ wafer will fit ˜46 devices); Silicon OxyNitride materials have excellent light transmission from 200 to 800 nm, and SiON can be deposited on a wafer with a low pressure chemical vapor deposition process.

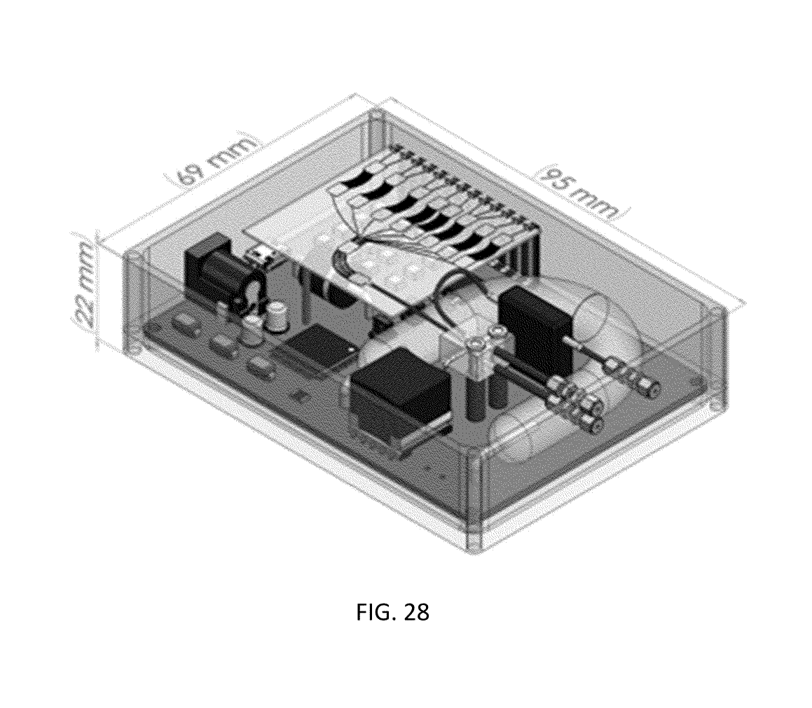

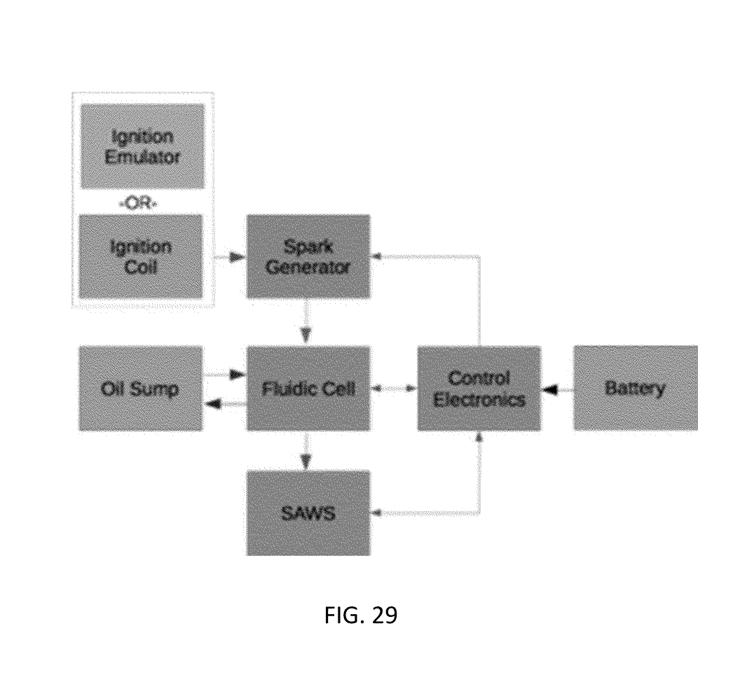

In certain embodiments, the invention encompasses an integrated MEMS product system as illustrated in FIG. 28 including:

compact fluidic cell w/ oil jet wetting of electrodes;

electrode material choice & optimization to achieve 100 k sparks with target LOD's;

micro-fabricated compact SAWS spectrometer;

HV system scavenging from automotive ignition system;

fluidic cell w/ mm scale electrodes;

Compact SAWS spectrometer;

HV driver based on auto ignition coil (preferred) or 2-stage capacitive pulse;

approximately 140 cc.

TABLE 2
Illustrative Elements of Interest for Detection
Elements of Interest & Corresponding
Wavelength of Occurrence

| | | | | Wavelength |
|---|---|---|---|---|
| | | | Element | (nm) |
| | | 1 | Zinc | 202.52 |
| Bin 1 | {open oversize brace} | | | |
| | | 2 | Phosphorus | 214.92 |
| | | 3 | Manganese | 257.61 |
| Bin 2 | {open oversize brace} | | | |
| | | 4 | Iron | 259.95 |
| | | 5 | Magnesium | 279.56 |
| Bin 3 | {open oversize brace} | | | |
| | | 6 | Silicon | 288.16 |
| Bin 4 | | 7 | Copper | 324.75 |
| Bin 5 | | 8 | Nickel | 352.41 |
| | | 9 | Molybdenum | 390.29 |
| Bin 6 | {open oversize brace} | 10 | Calcium | 393.37 |
| | | 11 | Aluminum | 396.15 |
| Bin 7 | | 12 | Sodium | 588.99 |
| Bin 8 | | 13 | Potassium | 766.49 |

### Single Integrated Optical/Microfluidics Oil Apparatus

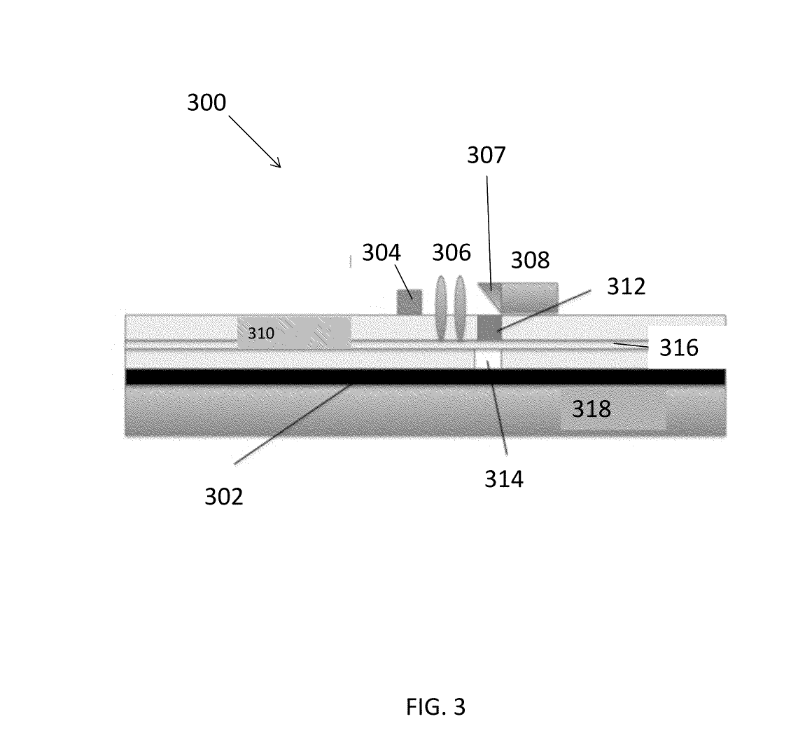

In the integrated optical/microfluidics apparatus 300 shown in FIG. 3, oil flow fluidic channels 302 (i.e., microfluidic flow channels), a laser source 304 (e.g. diode pump), focusing optics 306 (e.g, lenses), a reflector 307, and collection optics to gather light emitted by the plasma generated by the laser 308 are integrated onto a single integrated optical bench 310 oriented on a substrate 318 (e.g., a MEMS substrate form factor), along with a Yag crystal 312, and window 316 through which to view the air gap 314 and the oil below. This workbench contains precision-defined mechanical alignment features created with photolithography (micro-fabrication) or other high precision techniques. Because microfluidic channels can be fabricated reliably with features on the order of ten microns or less, and mechanical alignment features can place components (excitation and collection optics, detectors) with a similar degree of precision, the sample can be brought reliably into close alignment with the excitation laser focus spot, and the collection optics, which in turn can be accurately aligned to the detector. This approach provides multiple advantages over separately aligned free-space optics and fluidic components. Specifically, by bringing all components into close proximity, and by locking all components to a precision fabricated monolithic optics bench, the usual trouble with hand aligning separate optical components is eliminated.

Filter Actuator Target with Backflow

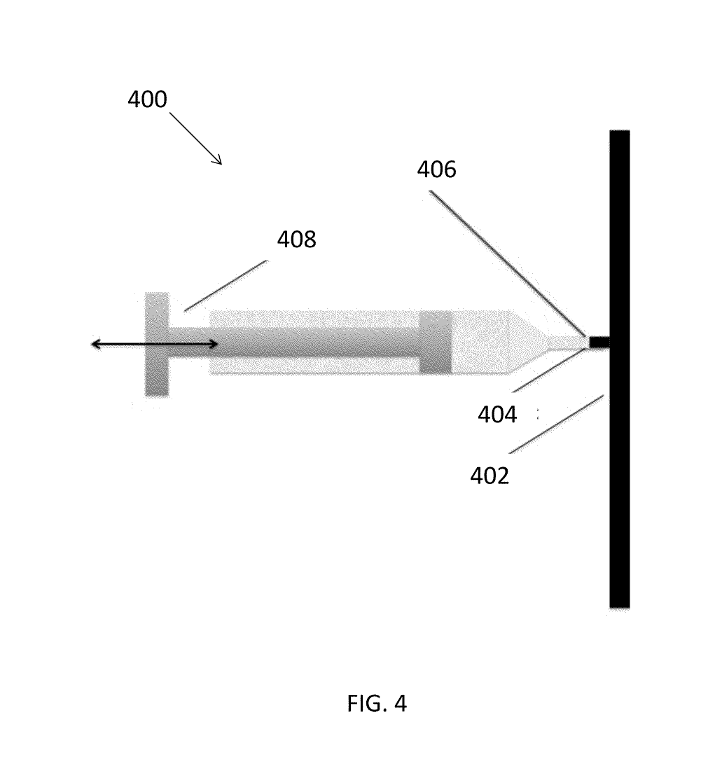

An additional approach in FIG. 4, showing filter actuator 400, is to create an oil sample matrix that effectively locks an oil sample 402 to a defined space. For example, a porous filter or trap element 404 through which oil is withdrawn, and the filter pulled against a mechanical backstop. The ending location of the filter surface is designed to be inside the optimal focal point of the excitation laser 406. This approach has the advantage that it presents not only the oil sample to be analyzed, but also particulates that may be trapped in the oil. Analysis of the particulate matter could potentially form an important component of the oil wear monitoring system. By flowing the sample in reverse (back-flowing) the particulate matter could be released. A simple valve structure could direct the flow of exposed sample to a waste stream or back to the main oil circulation (and oil filter.) For example, a solid plug of filter material is drawn or pulled through a plunger or piston structure 408 for analysis by LIBS. The piston draws a fluidic sample from the main circulation into a small channel, where the oil passes through a porous filter, which captures oil and contaminant particles. The excitation laser is focused on the surface of the filter. Reversal of piston motion reverses fluid flow and pushes analyzed sample and particles back into main flow.

### Droplet Ejection Architectures

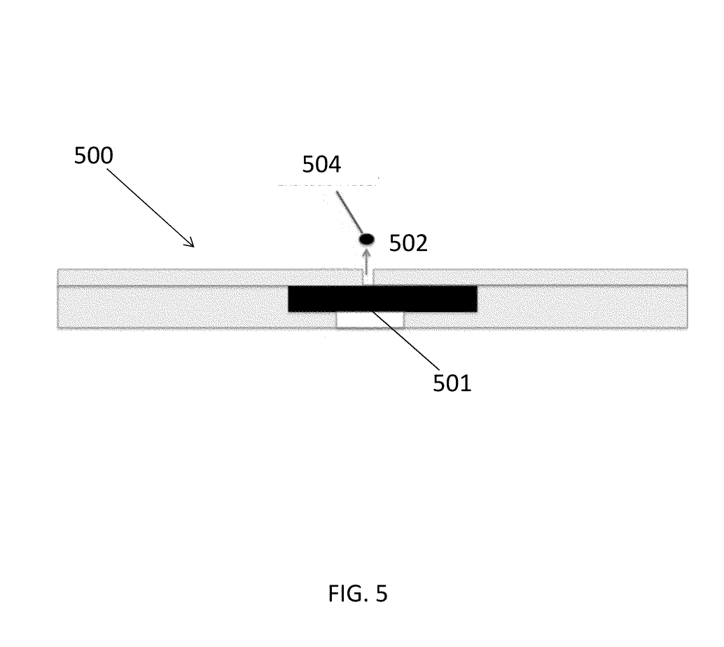

In FIG. 5, a microdroplet injection apparatus 500 is shown. A small reservoir of liquid, e.g., oil 501 to be analyzed, is created, and then a droplet is ejected 502 into a focal point of excitation laser 504. The droplet is suitably ejected by an apparatus that generates electrostatic, acoustic (ultrasonic) or other suitable method to generate the liquid droplet.

### Focused Jet Approach

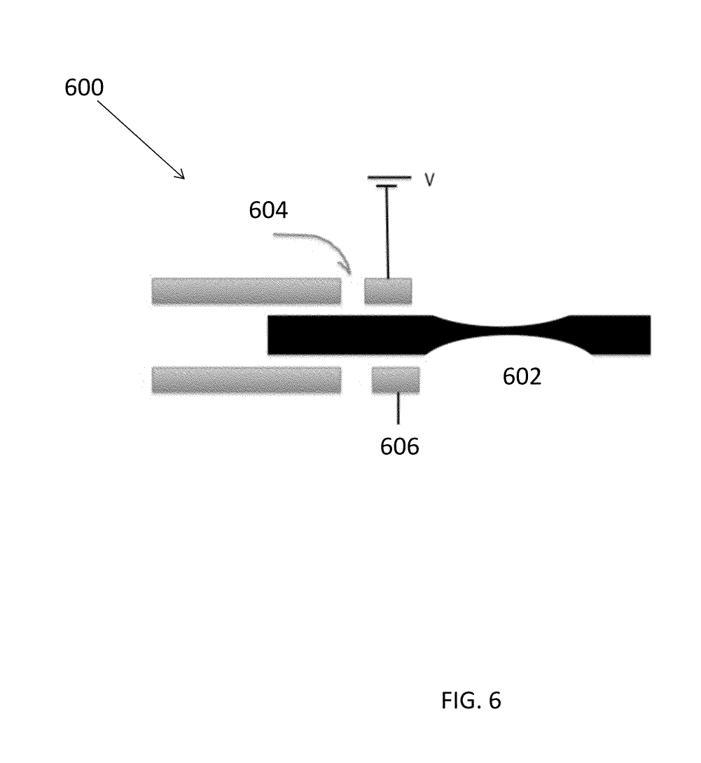

FIG. 6 shows a focused jet application 600 as described herein. An oil stream with a very narrow cross sectional area 602 can be created by a focusing apparatus, such as a hydrodynamic focusing 604 (gas or liquid sheath flow) and/or the application of an electric potential between the fluid and an external electrode 606. In addition, the position of the stream can be deflected by adjusting sheath flow and voltage parameters, creating the possibility that the stream can be deflected with positive feedback until it is located within the laser focal point. By preparing a tightly focused stream, the narrow stream becomes a string of droplets as in electrospray injection in mass spectrometry.

These methods and apparatuses described herein can be applied in any situation in which it is desired to monitor the composition of a liquid (such as oil) in a harsh environment (industrial, automotive, aviation) from an inherently size-limited platform. Examples include the monitoring of liquids found in: transmissions, aircraft rotors, transformer and other industrial equipment, as well as the characterization of food oil and other foodstuffs, chemical composition in the drug and pharmaceutical development process, and the detection of chemical and biological agents in effluent waste streams for environmental monitoring, homeland security and defense.

### Example 1

### Analysis of Oil Samples

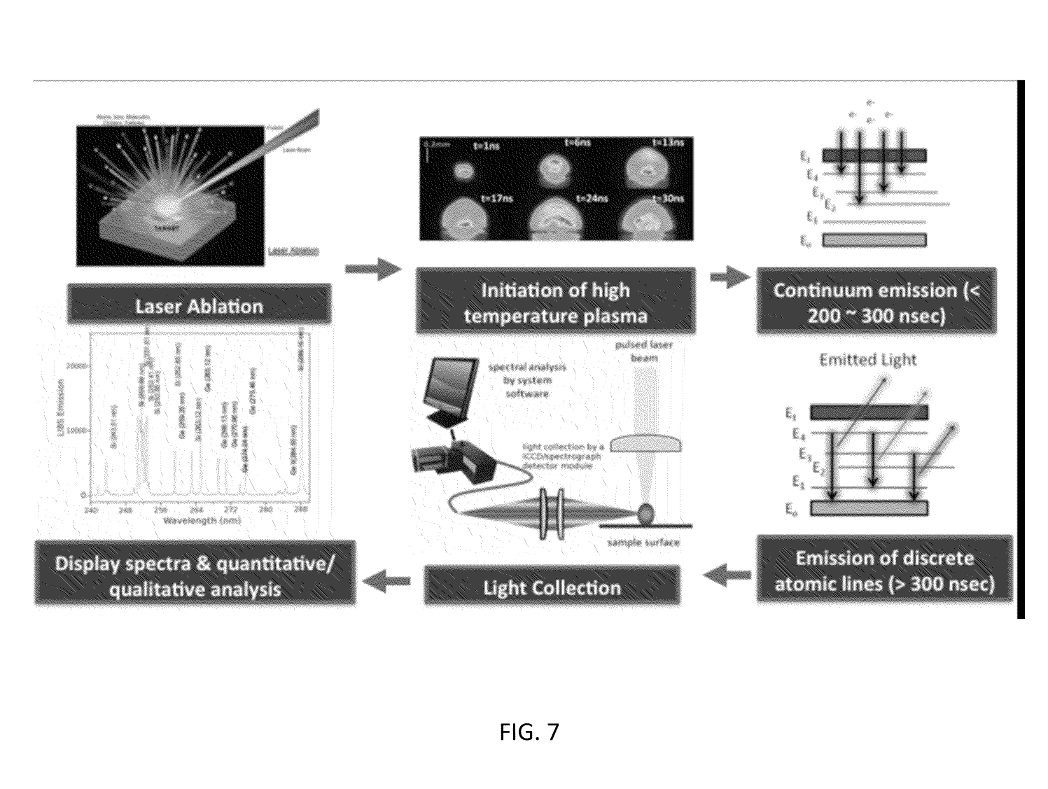

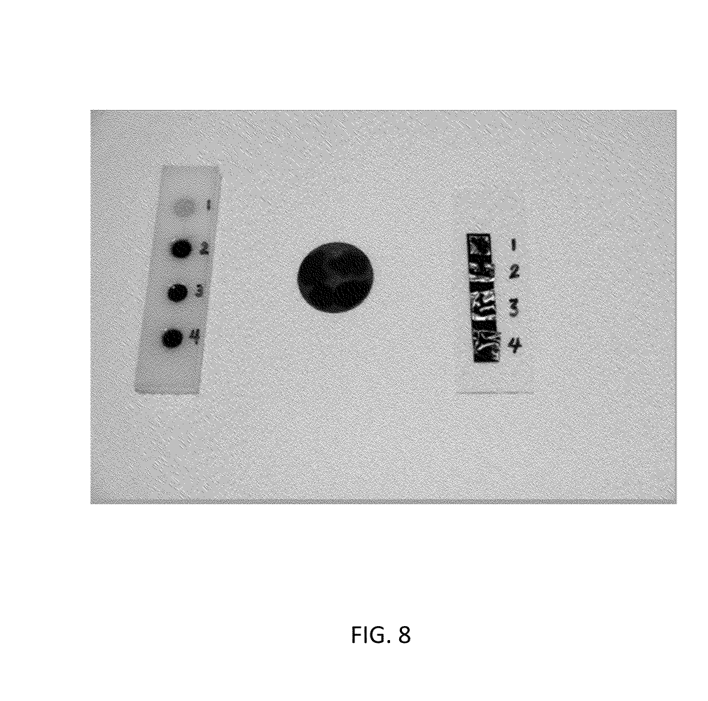

LIBS analysis was performed using a J200 LIBS instrument manufactured by Applied Spectra, Inc. of Fremont Calif. A 266 nm UV laser was used to ablate oil samples and multiple laser pulses were used to provide an indication of elemental composition variations. Samples were prepared that included the oil materials, the oils absorbed into a graphite surface and the oils after thermal decomposition to concentrate the elements present. Several thousand spectra were collected with variations in the laser power used, laser spot size, analyzer delay time, sample disposition (exposed, covered with weigh paper, analyzed through an aperture, absorbed onto a solid media, thermally decomposed). After data collection, comparisons were made among the collected spectra with particular attention to the comparison of data from samples that had the greatest dispersion of concentrations for the wear elements. A flowchart showing the general operation of LIBS is shown in FIG. 7. FIG. 8 shows samples mounted for analysis as liquid (left), absorbed into graphite (center) and heated to concentrate (right).

Table 1 is a tabulation of the elemental concentrations (wear elements) for the element set as determined by spectroscopic analysis performed in accordance with ASTM Method D5185 “Standard Test Method for Multi-element Determination of Used and Unused Lubricating Oils and Base Oils by Inductively Coupled Plasma Atomic Emission Spectrometry (ICP-AES)”. From these data, a comparison is made between Sample #1 with a smaller contribution from expected wear products and Sample #2 with a higher contribution to demonstrate the utility of using LIBS measurement and analysis.

TABLE 1
TESTOIL REFERENCE RESULTS
Spectroscopic Analysis (ppm) ASTM D5185

| Element | Sample 1 | Sample 2 | Sample 3 | Sample 4 |
|---|---|---|---|---|
| Iron (Fe) | 0 | 124 | 17 | 8 |
| Copper (Cu) | 0 | 199 | 8 | 0 |
| Lead (Pb) | 0 | 8 | 1 | 0 |
| Aluminum (Al) | 0 | 19 | 2 | 1 |
| Tin (Sn) | 2 | 5 | 3 | 2 |
| Nickel (Ni) | 0 | 0 | 0 | 0 |
| Chromium (Cr) | 0 | 0 | 0 | 0 |
| Titanium (Ti) | 0 | 0 | 0 | 0 |
| Vanadium (V) | 0 | 0 | 0 | 0 |
| Silver (Ag) | 0 | 0 | 0 | 0 |
| Silicon (Si) | 2 | 248 | 22 | 7 |
| Boron (B) | 225 | 4 | 27 | 8 |
| Calcium (Ca) | 1825 | 2047 | 1567 | 1681 |
| Magnesium (Mg) | 19 | 15 | 25 | 16 |
| Phosphorus (P) | 587 | 752 | 667 | 684 |
| Zinc (Zn) | 735 | 872 | 770 | 844 |
| Barium (Ba) | 0 | 10 | 0 | 0 |
| Molybdenum (Mo) | 75 | 553 | 288 | 329 |
| Sodium (Na) | 4 | 18 | 255 | 315 |
| Potassium (K) | 0 | 0 | 0 | 0 |

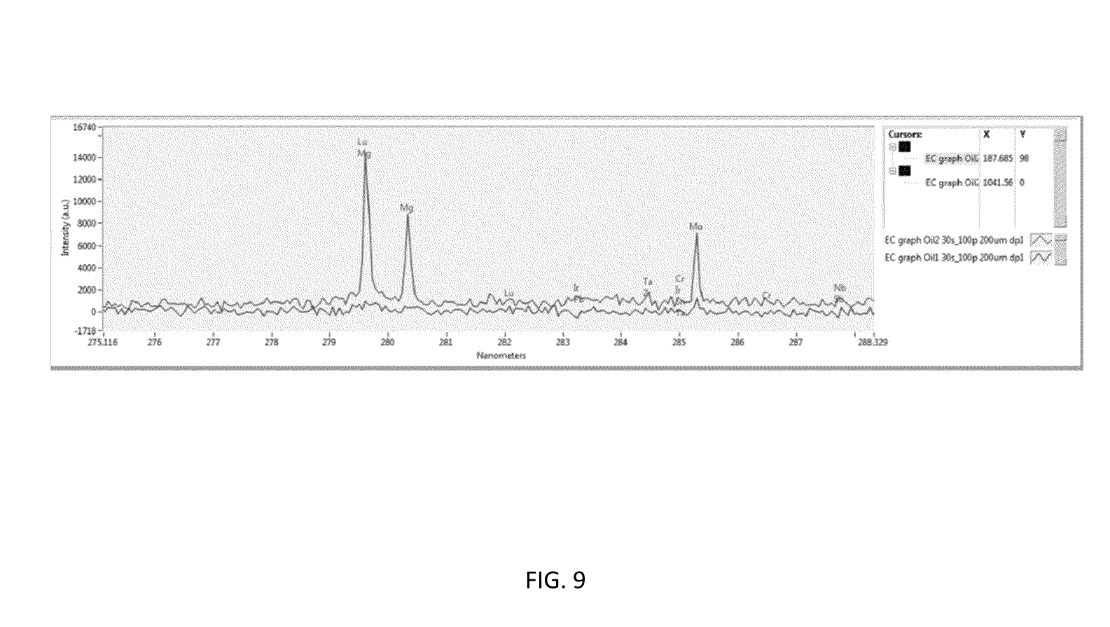

FIG. 9 shows data collected from the Sample #1 and #2 oils that had been absorbed into a graphite matrix. The upper plot is Sample #2 (high wear) showing elevated Mg and Mo compared with Sample #1. This method of analysis greatly enhanced the signals obtained from Zn, Na and K relative to the other elements and provided a base to absorb a major portion of the laser thermal energy.

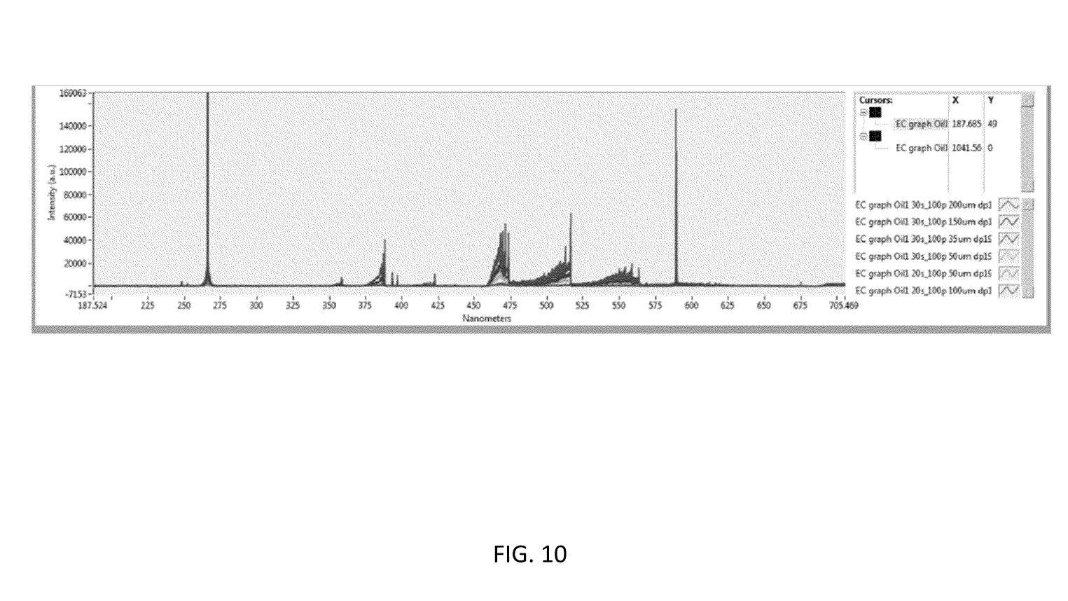

FIG. 10 is a comparison of the Sample #1 and #2 oils using varied laser spot sizes to select the most efficient signal production parameter.

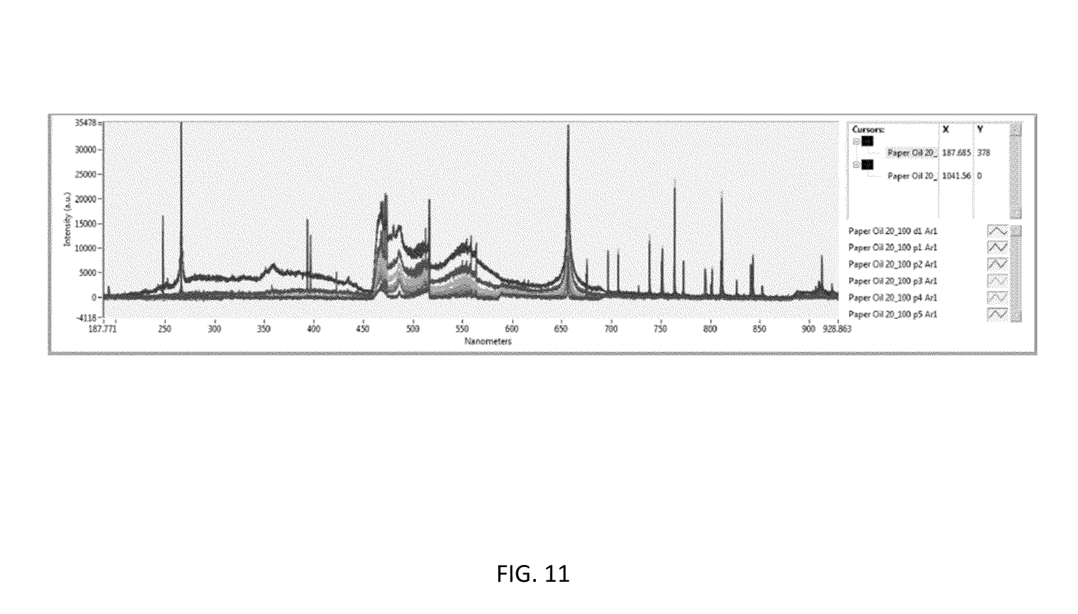

FIG. 11 shows a comparison of various analyzer delay times from 0.1 ms to 1 msec. A delay time of 0.195 msec was selected as producing the highest sensitivity for the wear elements with the attenuation of signals from H, C and S from the oil matrix.

Additional results were obtained through a platinum aperture laid across the oil wells drilled into a Teflon® strip that was used to contain the oils for analysis.

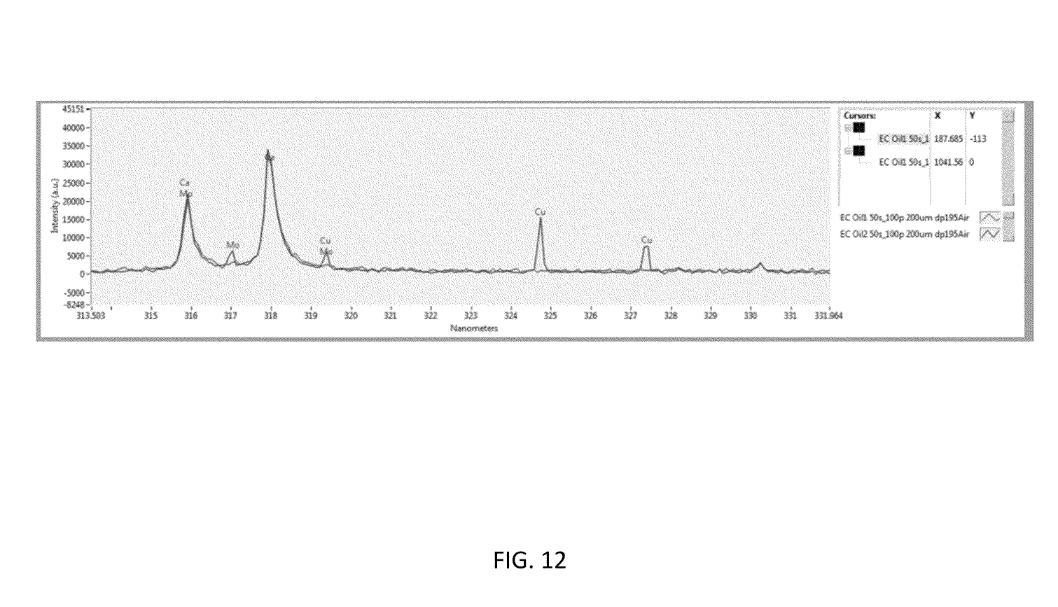

FIG. 12 shows an overlay of Samples #1 and #2 through a Pt aperture. The analyzer delay is 0.195 msec, 200 μm laser size. Sample #2 (high wear) showed elevated Cu and Mo compared with Sample #1. Ca was similar in intensity on the two samples as suggested by ICP-AES

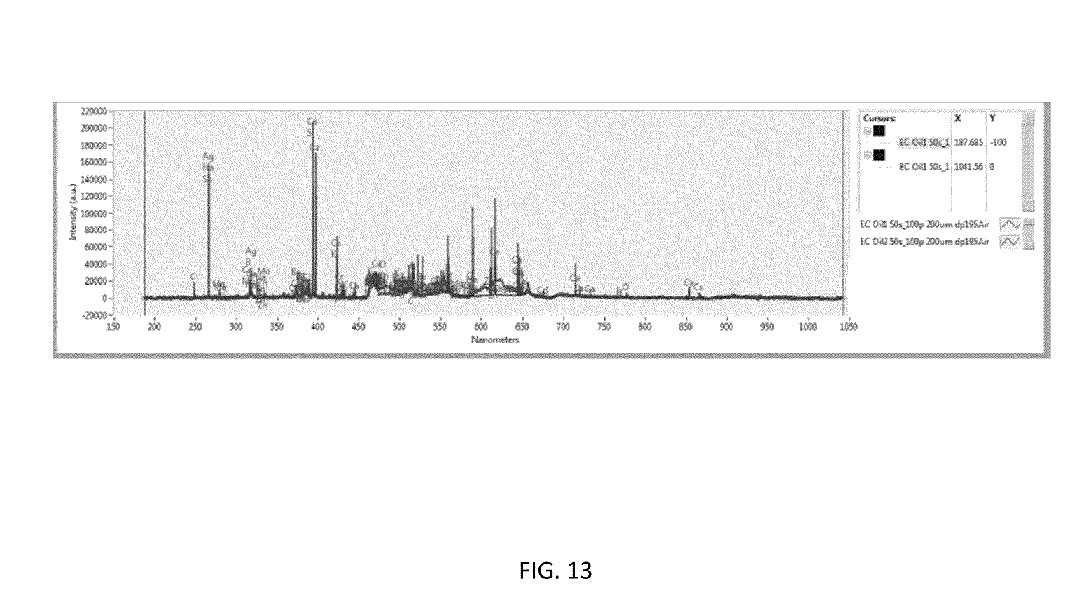

FIG. 13 shows an additional overlay of Samples #1 and #2 through a Pt aperture. The analyzer delay is 0.195 msec, 200 μm laser size. Sample #2 (high wear) showed elevated wear materials compared with Sample #1.

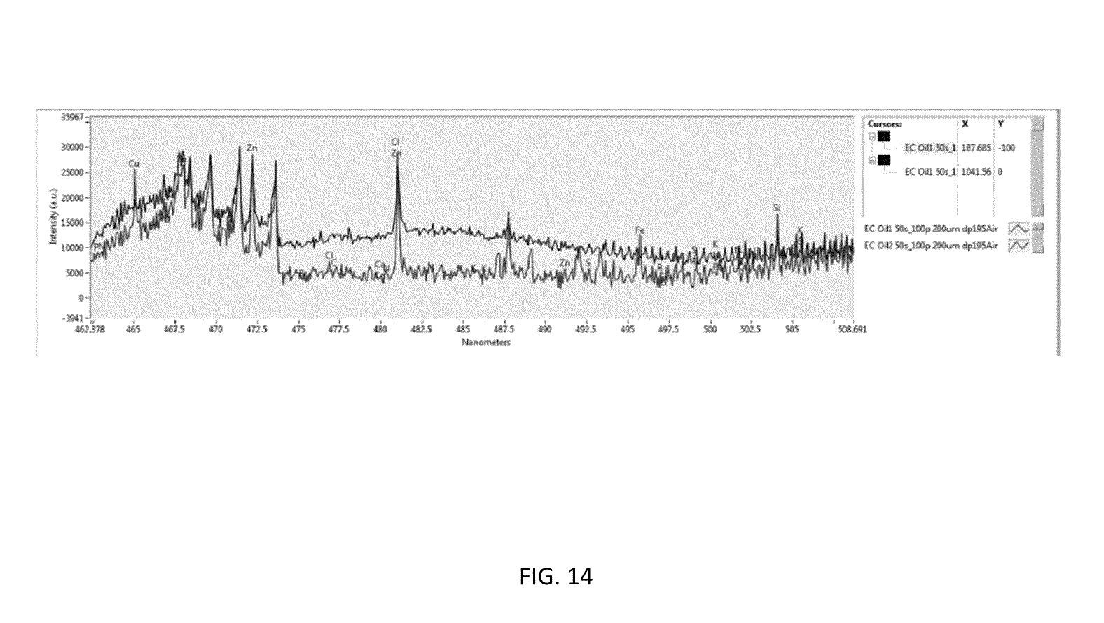

FIG. 14 shows an overlay of Sample #1 and #2 though Pt aperture. The analyzer delay is 0.195 msec, 200 μm laser size. Sample #2 (High Wear) shows elevated Si and Fe compared with Sample #2. Zn is similar in intensity in the two samples as suggested by ICP-AES.

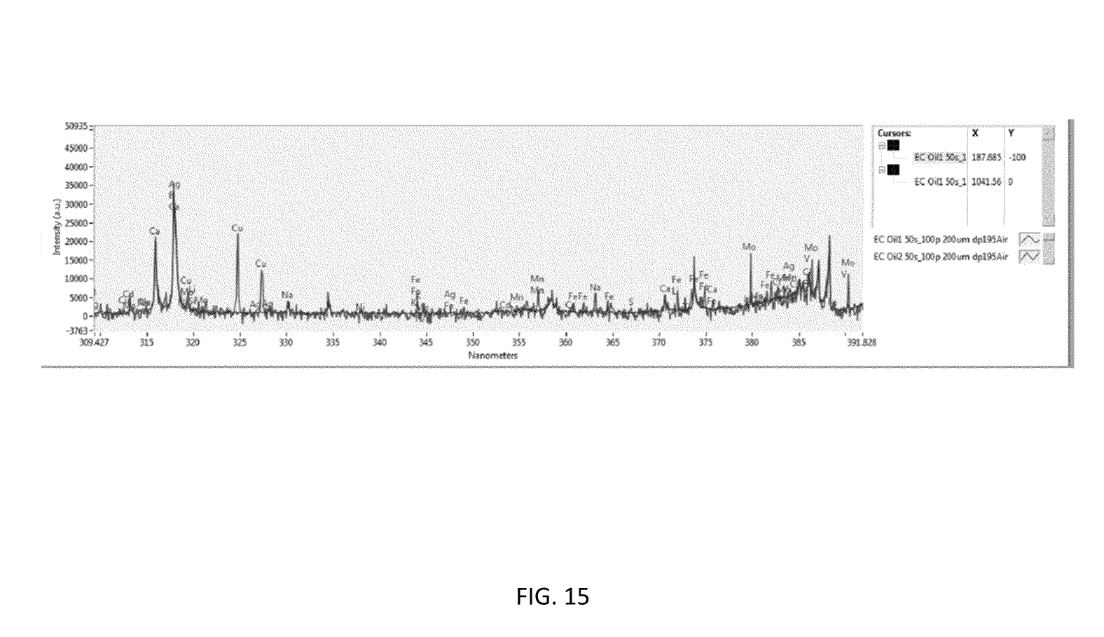

FIG. 15 shows an overlay of samples #1 and #2 through Pt aperture. The analyzer delay is 0.195 msec, 200 μm laser size. Sample #2 (High wear) shows elevated Na, Cu, Mo, Mn and Fe compared with Sample #1. Ca is similar in intensity on the two samples as suggested by ICP-AES.

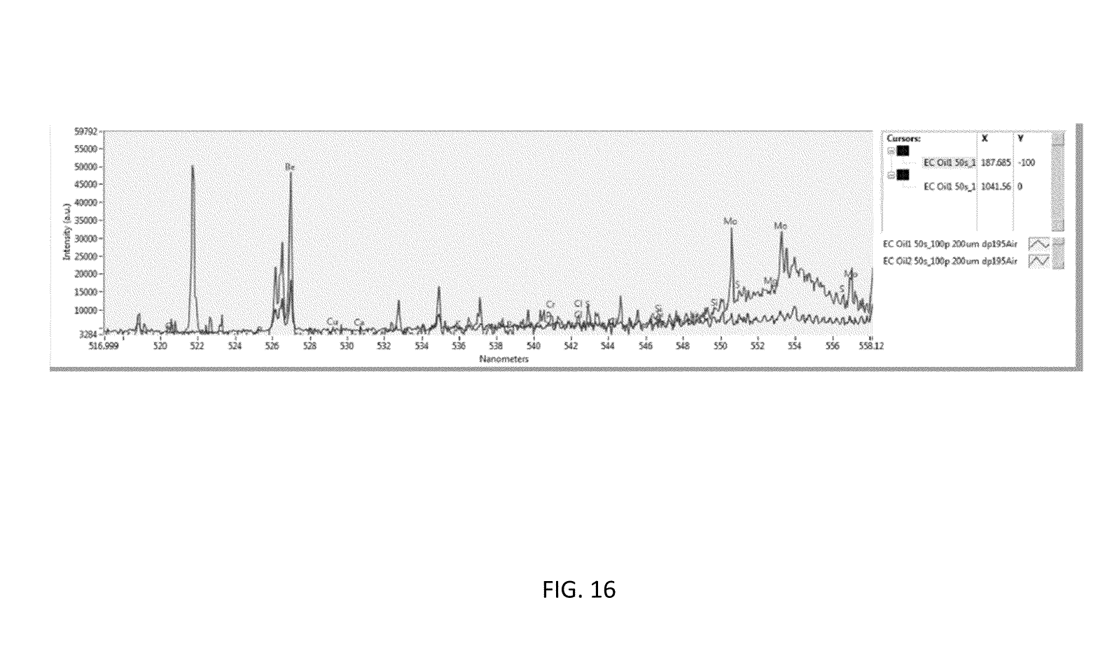

FIG. 16 shows an overlay samples #1 and #2 through Pt aperture. The analyzer delay is 0.195 msec, 200 μm laser size. Sample #2 (high wear) shows elevated Mo, Cr and Be compared to Sample #1.

The foregoing description, for purpose of explanation, has been described with reference to specific embodiments. The illustrative discussions above, however, are not intended to be exhaustive or to limit the invention to the precise forms disclosed. Many modifications and variations are possible in view of the above teachings. The embodiments were chosen and described in order to explain the principles of the invention and its practical applications, to thereby enable others skilled in the art to best utilize the invention and various embodiments with various modifications as are suited to the particular use contemplated.

What is claimed is:

1. An integrated micro-electromechanical (MEMS) breakdown spectroscopy apparatus for detecting a wear element in a liquid, the apparatus comprising:
a. a MEMS substrate form factor;
b. a breakdown means integrated with the MEMS substrate form factor;
c. means to generate a plasma;
d. a spectrometer configured to measure a spectrum of light emitted by the plasma and produce data regarding the wear element; and
e. electronics for transmitting the data regarding the wear element.

2. The integrated MEMS apparatus of claim 1, wherein the breakdown means is laser induced breakdown.

3. The integrated MEMS apparatus of claim 1, wherein the breakdown means is spark induced breakdown.

4. The integrated MEMS apparatus of claim 1, wherein the spectrometer is a selective arrayed waveguide spectrometer.

5. The integrated MEMS apparatus of claim 1, wherein the spectrometer is a Czerny-Turner (CT) spectrometer.

6. The integrated MEMS apparatus of claim 1, wherein the liquid is an oil-based lubricant.

7. The integrate MEMS apparatus of claim 2, wherein the laser is an IR laser.

8. The integrated MEMS apparatus of claim 7, wherein the laser is a sub-nanosecond pulse laser.

9. The integrated MEMS apparatus of claim 1, wherein the form factor is between about 30 cm³ and about 100 cm³.

10. The integrated MEMS apparatus of claim 6, wherein the oil-based lubricant is selected from the group consisting of:
a. an automotive lubricant;
b. a marine lubricant;
c. an aircraft lubricant;
d. an industrial device lubricant;
e. a compressor lubricant; and
f. a wind turbine lubricant.

11. The integrated MEMS apparatus of claim 1, wherein the wear element is selected from the group consisting of:
a. Na;
b. Mg;
c. Al;
d. Si;
e. Mn;
f. Fe;
g. Ni;
h. Cu;
i. Zn; and
j. Mo.

12. The integrated MEMS apparatus of claim 1, wherein the wear element in the liquid is detected at a level of between 0.1 and 200 parts per million.

13. A system comprising the MEMS apparatus of claim 1, and further comprising a receiver unit remotely located from the MEMS form factor.

14. A machine comprising the MEMS apparatus of claim 1.

15. The machine of claim 10, wherein the machine is selected from the group consisting of a car, a truck, a boat, a ship, an aircraft, an industrial machine, a compressor, and a wind turbine.

16. A method of detecting wear elements in a liquid, comprising:
a. providing a liquid sample;
b. contacting the liquid sample with a means to generate a plasma; and
c. detecting one or more wear elements in the plasma with laser-induced breakdown spectroscopy,
wherein the liquid is contacted with a laser that is integrated into a MEMS form factor.

17. The method of claim 16, wherein the liquid is an oil-based lubricant.

18. The method of claim 16, wherein the one or more wear elements are wear metals.

19. The method of claim 16, further comprising transmitting data regarding the one or more wear elements to a receiver remotely located from the MEMS form factor.

20. An integrated micro-electromechanical (MEMS) spark-induced breakdown spectroscopy (SIBS) apparatus for detecting a wear element in a liquid, the apparatus comprising:
a. a MEMS substrate form factor;
b. high voltage source connected to electrodes incorporated with the MEMS substrate form factor;
c. one or more focusing optics or reflectors;
d. a microfluidic flow channel comprising the liquid;
e. collection optics to gather light emitted by the liquid generated by the spark; and
f. a spectrometer configured to measure the spectrum of the light emitted by the plasma generated by the laser and generate data regarding the wear element.

21. The integrated MEMS SIBS apparatus of claim 16, wherein the liquid is an oil-based lubricant.

22. The integrated MEMS SIBS apparatus of claim 21, wherein the oil-based lubricant is selected from the group consisting of:
a. an automotive lubricant;
b. a marine lubricant;
c. an aircraft lubricant;
d. an industrial device lubricant;
e. a compressor lubricant; and
f. a wind turbine lubricant.

23. The integrated MEMS SIBS apparatus of claim 20, wherein the wear element is selected from the group consisting of:
a. Na;
b. Mg;
c. Al;
d. Si;
e. Mn;
f. Fe;
g. Ni;
h. Cu;
i. Zn; and
j. Mo.
